(19) United States
(12) Patent Application Publication
(10) Pub. No.: US 2012/0060413 A1
(43) Pub. Date: Mar. 15, 2012

(54) INCREASING CARBON FLOW FOR POLYHYDROXYBUTYRATE PRODUCTION IN BIOMASS CROPS

(75) Inventors: Mariya Somleva, Cambridge, MA (US); Himani Chinnapen, Quincy, MA (US); Aminat Ali, Cambridge, MA (US); Kristi D. Snell, Belmont, MA (US); Oliver P. Peoples, Arlington, MA (US); Nii Patterson, Chelmsford, MA (US); Jihong Tang, Brighton, MA (US); Karen Bohmert-Tatarev, Brookline, MA (US)

(73) Assignee: Metabolix, Inc.

(21) Appl. No.: 13/233,498

(22) Filed: Sep. 15, 2011

Related U.S. Application Data
(60) Provisional application No. 61/383,142, filed on Sep. 15, 2010.

(51) Int. Cl. C10L 8/00; C12N 5/10; C08L 67/04; C12N 15/82; C08L 97/02; A01H 5/00; A01H 5/10

(52) U.S. Cl. 44/605; 800/298; 435/419; 800/320; 800/278; 800/288; 524/14

(57) ABSTRACT

Transgenic plants, transgenic plant material, and transgenic plant cells for the improved synthesis of polyhydroxyalkanoates, preferably poly(3-hydroxybutyrate) (also referred to as PHB), have been developed. In one embodiment, carbon flow is modulated to increase production of PHB. Preferred plants that can be genetically engineered to produce PHB include plants that produce a large amount of lignocellulosic biomass that can be converted into biofuels, such as switchgrass, Miscanthus, Sorghum, sugarcane, millets, Napier grass and other forage and turf grasses. An exemplary plant that can be genetically engineered to produce PHB and produces lignocellulosic biomass is switchgrass, Panicum virgatum L. A preferred cultivar of switchgrass is Alamo. Other suitable cultivars of switchgrass include, but are not limited to, Blackwell, Kanlow, Nebraska 28, Pathfinder, Cave-in-Rock, Shelter and Trailblazer.

### CROSS REFERENCE TO RELATED APPLICATION

This application claims the benefit of U.S. Provisional Application No. 61/383,142, filed on Sep. 15, 2010. The entire disclosure of the above application is incorporated herein by reference.

### FIELD OF THE INVENTION

The invention is generally related to agricultural biotechnology, in particular to transgenic plants that produce polyhydroxyalkanoates.

### BACKGROUND OF THE INVENTION

Polyhydroxyalkanoates (PHAs), a family of naturally renewable and biodegradable plastics are an ideal value-added co-product in bioenergy crops slated for processing into liquid fuels and/or energy (Snell & Peoples, (2009), Biofuels Bioprod Bioref 3:456-467). These polymers occur in nature as a storage reserve in some microbes faced with nutrient limitation (Madison et al., (1999) Microbiol Mol Biol Rev 63:21-53) and possess properties enabling their use in a variety of applications currently served by petroleum-based plastics. Since PHAs are inherently biodegradable in soil, compost, and marine environments, they can decrease plastic waste disposal issues. Pathways for production of PHAs have been introduced into a number of crops [for review, see Suriyamongkol et al., (2007), Biotechnol Adv, 25:148-175 and Snell & Peoples, (2009), Biofuels Bioprod Bioref., 3:456-467 and references therein] including maize (Poirier et al., (2002), Biopolymers: Polyesters I—Biological Systems and Biotechnological Production (Doi Y and Steinbiichel A eds): 401-435, Weinheim: Wiley-VCH), sugarcane (Petrasovits et al., (2007), Plant Biotechnol J, 5:162-172, Purnell et al., (2007), Plant Biotechnol J, 5:173-184), flax (Wrobel-Kwiatkowska et al., (2007), Biotechnol Prog, 23:269-277, Wrobel et al., (2004), J. Biotechnol, 107:41-54), cotton (John et al., (1996), Proc Natl Acad Sci USA 93:12768-12773), alfalfa (Saruul et al., (2002), Crop Sci, 42:919-927), tobacco (Arai et al., (2001), Plant Biotechnol J, 18:289-293, Bohmert et al., (2002), Plant Physiol, 128:1282-1290, Lossl et al., (2005), Plant Cell Physiol, 46:1462-1471, Lössl et al., (2003), Plant Cell Rep, 21:891-899), potato (Bohmert et al., (2002), Plant Physiol, 128:1282-1290), and oilseed rape (Houmiel et al., (1999), Planta, 209:547-550, Slater et al., (1999), Nat Biotechnol, 17:1011-1016, Valentin et al., (1999), Int J Biol Macromol, 25:303-306) resulting in the production of a range of polymer levels depending on the crop and mode of transformation. See also U.S. Pat. Nos. 5,663,063 to Peoples et al., and 5,534,432 to Peoples. In switchgrass, PHB levels of 3.72% dry weight have been observed in samples of leaf tissue and 1.23% dry weight in the entire plant (Somleva et al., (2008), Plant Biotechol J, 6:663-678; U.S. 2009/0271889 A1). Higher PHB levels (up to 6.09% in mature leaves) have been measured in switchgrass plants propagated under in vitro conditions from primary transformants (WO 2010102220 A1; U.S. 2010/0229256 A1).

Switchgrass is one of the bioenergy crops targeted by the United States Department of Energy for development (DOE (2006), U.S. Department of Energy Office of Science and Office of Energy Efficiency and Renewable Energy (www.doegenomestolife.org/biofuels/), Sanderson et al., (2006), Can J Plant Sci, 86:1315-1325). Recent studies suggest that production of cellulosic ethanol from this crop nets 540% more renewable energy than the required nonrenewable energy inputs (Schmer et al., (2008), Proc Natl Acad Sci USA, 105:464-469).

### SUMMARY OF THE INVENTION

The transgenic plants and transgenic plant cells with pathways to increase carbon flow in biomass crops, such as switchgrass, for the production of polyhydroxyalkanoate (PHA) are provided. One embodiment provides transgenic plants or transgenic plant cells genetically engineered to produce PHA and to have increased lignocellulosic biomass relative to a corresponding non-genetically-engineered plant or plant cell. Methods and constructs for producing the transgenic plants and transgenic plant cells are also described. The transgenic plant or transgenic plant cell can include the NAD-malic enzyme photosynthetic pathway. It can further include one or more transgenes that increase carbon flow for the production of polyhydroxyalkanoates. The one or more transgenes can increase carbon flow through the Calvin cycle in photosynthesis. The one or more transgenes can be selected from the group consisting of sedoheptulose 1,7-bisphosphatase (SBPase, EC 3.1.3.37), fructose 1,6-bisphosphatase (FBPase, EC 3.1.3.11), a bi-functional enzyme with both SBPase and FBPase activities, transketolase (EC 2.2.1.1), and aldolase (EC 4.1.2.13). The bifunctional enzyme can be selected from the group consisting of Ralstonia eutropha H16 (Accession number AAA69974), Synechococcus elongatus PCC 7942 (Accession numbers D83512 (SEQ ID NO: 2) and CP000100 (SEQ ID NO: 1)), Synechococcus sp. WH 7805 (Accession number ZP_{—}01124026), Butyrivibrio crossotus DSM 2876 (Accession number EFF67670), Rothia mucilaginosa DY-18 (Accession number YP_{—}003363264), Thiobacillus denitrificans ATCC 25259 (Accession number AAZ98530), Methylacidiphilum infernorum V4 (Accession number ACD83413), Nitrosomonas europaea ATCC 19718 (Accession number CAD84432), Vibrio vulnificus CMCP6 (Accession number AA009802), and Methanohalophilus mahii DSM 5219 (Accession number YP_{—}003542799). The plant or plant cell that is transformed to produce the transgenic plant or transgenic plant cell can be selected from the group consisting of switchgrass, Miscanthus, Sorghum, sugarcane, energy cane, giant reed, millets, Napier grass, other forage grasses and turf grasses. More specifically, the plant can be the switchgrass Panicum virgatum L. The plant can be a cultivar of switchgrass, such as Alamo, Blackwell, Kanlow, Nebraska 28, Pathfinder, Cave-in-Rock, Shelter and Trailblazer. The plant or plant cell that is transformed to produce the transgenic plant or transgenic plant cell can be a C₄ plant. The transgenic plant can produce at least about 4%, at least about 5%, at least about 6%, at least about 7%, at least about 8% dry weight (dwt) polyhydroxyalkanoate.

Also provided, are transgenic plants produced from such transgenic plants or transgenic plant cells, and seeds obtained from such transgenic plants or transgenic plant cells.

In addition, provided herein is a feedstock composition for production of biofuel, pyrolysis liquids, syngas, steam power or cogeneration power, where the feedstock includes at least about 3 to about 7.7% PHB and lignocellulosic biomass.

Also provided is a feedstock composition for production of biofuel, pyrolysis liquids, syngas, steam power or cogeneration power, where the feedstock includes at least about 3 to about 7.7% PHB and lignocellulosic biomass with modified structural carbohydrates.

Either of these feedstock compositions can be obtained from the transgenic plants or plant parts provided herein.

Provided herein is a method for increasing carbon flow through the Calvin cycle in photosynthesis, where the method includes: providing embryogenic callus cultures initiated from a transgenic plant; introducing into the embryogenic callus cultures transgenes that increase carbon flow through the Calvin cycle (selected from the group consisting of sedoheptulose 1,7-bisphosphatase (SBPase, EC 3.1.3.37), fructose 1,6-bisphosphatase (FBPase, EC 3.1.3.11), a bi-functional enzyme with both SBPase and FBPase activities, transketolase (EC 2.2.1.1), and aldolase (EC 4.1.2.13)), thereby producing re-transformed callus cultures; and regenerating plants from the re-transformed callus cultures, thereby producing plants with increased carbon flow through the Calvin cycle in photosynthesis. The bifunctional enzyme can be selected from the group consisting of Ralstonia eutropha H16 (Accession number AAA69974), Synechococcus elongatus PCC 7942 (Accession numbers D83512 (SEQ ID NO: 2)and CP000100 (SEQ ID NO: 1)), Synechococcus sp. WH 7805 (Accession number ZP_{—}01124026), Butyrivibrio crossotus DSM 2876 (Accession number EFF67670), Rothia mucilaginosa DY-18 (Accession number YP_{—}003363264), Thiobacillus denitrificans ATCC 25259 (Accession number AAZ98530), Methylacidiphilum infernorum V4 (Accession number ACD83413), Nitrosomonas europaea ATCC 19718 (Accession number CAD84432), Vibrio vulnificus CMCP6 (Accession number AA009802), and Methanohalophilus mahii DSM 5219 (Accession number YP_{—}003542799). The embryogenic callus culture can be derived from a plant selected from the group consisting of switchgrass, Miscanthus, Sorghum, sugarcane, energy cane, giant reed, millets, Napier grass, other forage grasses and turf grasses. The plant can be switchgrass (Panicum virgatum L.), or a cultivar of switchgrass. The cultivar of switchgrass can be selected from the group consisting of Alamo, Blackwell, Kanlow, Nebraska 28, Pathfinder, Cave-in-Rock, Shelter and Trailblazer. The embryogenic callus culture can be derived from a transgenic C₄ plant. In any of the methods provided, the plants with increased carbon flow through the Calvin cycle in photosynthesis can produce at least about 4%, at least about 5%, at least about 6%, at least about 7%, at least about 8% dry weight (dwt) polyhydroxyalkanoate.

Transgenic plants, plant material, and plant cells for the improved synthesis of polyhydroxyalkanoates, preferably poly(3-hydroxybutyrate) (also referred to as PHB), have been developed. Preferred plants that can be genetically engineered to produce PHB include plants that produce a large amount of lignocellulosic biomass that can be converted into biofuels, such as switchgrass, Miscanthus, Sorghum, sugarcane, energy cane, millets, Napier grass, giant reed, and other forage and turf grasses. An exemplary plant that can be genetically engineered to produce PHB and produces lignocellulosic biomass is switchgrass, Panicum virgatum L. A preferred cultivar of switchgrass is Alamo. Other suitable cultivars of switchgrass include, but are not limited to, Blackwell, Kanlow, Nebraska 28, Pathfinder, Cave-in-Rock, Shelter and Trailblazer.

In one embodiment, a plant, plant tissue, or plant material capable of producing lignocellulosic biomass is engineered to express genes encoding enzymes in the PHA biosynthetic pathway. The preferred PHA is PHB. Genes useful for production of PHB include phaA, phaB, and phaC, all of which are known in the art. The genes can be introduced in the plant, plant tissue, or plant cell using conventional plant molecular biology and transformation techniques.

Another embodiment provides a transgenic plant genetically engineered to produce at least about 4% dry weight (DW) polyhydroxyalkanoate. The polyhydroxyalkanoate content per unit dry weight can be at least about 4.0%, 4.1%, 4.2%, 4.3%, 4.4%, 4.5%, 4.6%, 4.7%, 4.8%, 4.9%, at least about 5.0%, 5.1%, 5.2%, 5.3%, 5.4%, 5.5%, 5.6%, 5.7%, 5.8%, 5.9%, at least about 6.0%, 6.1%, 6.2%, 6.3%, 6.4%, 6.5%, 6.6%, 6.7%, 6.8%, 6.9%, at least about 7.0%, 7.1%, 7.2%, 7.3%, 7.4%, 7.5%, 7.6%, 7.7%, 7.8%, 7.9%, at least about 8.0%, 8.1%, 8.2%, 8.3%, 8.4%, 8.5%, 8.6%, 8.7%, 8.8%, 8.9%, at least about 9.0%, 9.1%, 9.2%, 9.3%, 9.4%, 9.5%, 9.6%, 9.7%, 9.8%, 9.9%, or at least 10%. Preferably the polyhydroxyalkanoate is PHB, and the PHB content is between about 2% and about 10%, more preferably between about 3% and about 8%, or between about 3% and about 7.6%.

Preferably the transgenic plant is a C₄ plant with the NAD-malic enzyme photosynthetic pathway. A preferred transgenic plant is switchgrass engineered with heterologous genes encoding a thiolase, a reductase, and a PHA synthase, as well as one or more additional transgenes for increased carbon flow, for the production of poly(3-hydroxybutyrate). Additional transgenes encoding enzymes can be selected from the group capable of increasing carbon flow through the Calvin cycle in photosynthesis. Candidate enzymes include but are not limited to sedoheptulose 1,7-bisphosphatase (SBPase, EC 3.1.3.37), fructose 1,6-bisphosphatase (FBPase, EC 3.1.3.11), a bi-functional enzyme encoding both SBPase and FBPase, transketolase (EC 2.2.1.1), and aldolase (EC 4.1.2.13). SBPase, transketolase, and aldolase activities have been shown to have an impact on the control of carbon fixed by the Calvin cycle (Raines, (2003), Photosynth Res, 75:1-10) which could be attributed to an increase in ribulose 1,5-bisphosphate regenerative capacity. Ribulose 1,5-bisphosphate is the acceptor molecule in the Calvin cycle that upon fixation of CO₂, is converted to two molecules of 3-phosphoglycerate. Bifunctional enzymes that contain both FBPase and SBPase activities have been reported from for example Ralstonia eutropha H16 (Accession number AAA69974), Synechococcus elongatus PCC 7942 (Accession numbers D83512 (SEQ ID NO: 2) and CP000100 (SEQ ID NO: 1)), Synechococcus spp. WH 7805 (Accession number ZP_{—}01124026), Butyrivibrio crossotus DSM 2876 (Accession number EFF67670), Rothia mucilaginosa DY-18 (Accession number YP_{—}003363264), Thiobacillus denitrificans ATCC 25259 (Accession number AAZ98530), Methylacidiphilum infernorum V4 (Accession number ACD83413), Nitrosomonas europaea ATCC 19718 (Accession number CAD84432), Vibrio vulnificus CMCP6 (Accession number AA009802), and Methanohalophilus mahii DSM 5219 (Accession number YP_{—}003542799).

The FBPase/SBPase gene from Synechococcus elongatus PCC 7942 has previously been expressed in tobacco and enhanced both photosynthesis and plant growth (Miyagawa, (2001), Nat Biotechnol, 19:965-969). Expression of an Arabidopsis SBPase cDNA in tobacco also has resulted in greater biomass and increased photosynthetic capacity (Raines, (2003), Photosynth Res, 75:1-10; Lefebvre et al., (2005), Plant Physiol 138:451-460).

Another embodiment provides seeds of the disclosed transgenic plants. Another embodiment provides plants propagated through cell and tissue cultures from the disclosed transgenic plants and seeds from the in vitro propagated plants.

Another embodiment provides plants or plant parts that are capable of growth to produce a plant with large quantities of biomass. These plant parts include, but are not limited to, apical and axillary meristems, leaves, stem tissues, roots, inflorescences, crowns, rhizomes, seedlings, plantlets, etc.

Still another embodiment provides feedstock from the disclosed transgenic plants. The feedstock typically contains at least about 3 to about 7.7% PHB and lignocellulosic biomass from the plants.

Another embodiment provides a method for re-transforming transgenic lines with a gene construct with two or more expression cassettes. Typically, the transgenic plants are engineered for the production of PHB and their product yield and agronomic performance are well characterized.

It should be understood that this invention is not limited to the embodiments disclosed herein and includes modifications that are within the spirit and scope of the invention.

### BRIEF DESCRIPTION OF THE DRAWINGS

FIG. 1 is an alignment of the FBPase/SBPases from accession numbers CP000100 (SEQ ID NO: 1) and D83512 (SEQ ID NO: 2). Plant transformation vector pMBXS422 (SEQ IS NO: 3) contains a DNA sequence with 100% identity to CP000100 (SEQ ID NO: 1).

FIG. 2 shows a Western blot of total soluble proteins (12 μg per lane) incubated with an antibody against the FBPase/SBPase protein sequence. Protein isolation and membrane blotting were performed as described previously (Somleva et al., (2008), Plant Biotechnol J, 6:663-678). An Affinity-Purified Peptide Polyclonal Antibody was produced by GenScript using the peptide sequence LMGKGEKNEADRVA (SEQ ID NO: 4). Lanes: WT—a control, wild-type plant; 1 to 6—PHB producing plants re-transformed with pMBXS422 (SEQ ID NO: 3); M—ColorPlus Prestained Protein Molecular Weight Markers (10-230 kDa); PC—a positive control (cell lysate from E. coli transformed with plasmid pMBXS634 containing the CP000100 (SEQ ID NO: 1) sequence behind a promoter functional in E. coli). The polymer levels measured in the mature leaves of the transgenic plants 1 to 6 two months after transfer to soil were 2.08, 2.04, 6.41, 4.32, 2.81, and 5.25% DW, respectively.

FIG. 3 shows the profile of structural carbohydrates in total leaf biomass from control and re-transformed PHB producers. A—contents of glucan (white bars) and xylan (light shaded bars), B—contents of galactan (white bars), mannan (light shaded bars), and arabinan (dark shaded bars). PHB genes—a PHB producing plant regenerated from immature inflorescence-derived cultures (control); PHB genes+pMBXS422 A and PHB genes+pMBXS422 B—PHB producing plants regenerated from immature inflorescence-derived cultures re-transformed with pMBXS422 (SEQ ID NO: 3) representing independent re-transformation events. The PHB contents in the samples from total leaf biomass were 1.88% DW (control PHB producer), 2.27% DW (re-transformant A), and 2.08 (re-transformant B). The results are presented, on a dry weight basis, as a weight percentage of the biomass.

FIG. 4 illustrates a comparison of the activity of photosystem II (PSII) in re-transformed and control switchgrass plants. A—chlorophyll fluorescence, B—quantum yield, C—electron transport rate (ETR). All measurements were performed in light adapted leaves from vegetative tillers at the same growth stage using MONI-PAM. WT—a non-transformed plant; PHB genes—a PHB producing plant regenerated from immature inflorescence-derived cultures; PHB genes+pMBXS422−a PHB producing plant regenerated from immature inflorescence-derived cultures re-transformed with pMBXS422 (SEQ ID NO: 3). The PHB levels in mature leaves were 3.53% DW (the control PHB producer) and 6.28% DW (the PHB genes+pMBXS422 plant).

### DETAILED DESCRIPTION OF THE INVENTION

### I. Definitions

A number of terms used herein are defined and clarified in the following section.

The term “PHA copolymer” refers to a polymer composed of at least two different hydroxyalkanoic acid monomers.

The term “PHA homopolymer” refers to a polymer that is composed of a single hydroxyalkanoic acid monomer.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences.

As used herein, an “expression control sequence” is a DNA sequence that controls and regulates the transcription and/or translation of another DNA sequence.

As used herein, “operably linked” means incorporated into a genetic construct so that expression control sequences effectively control expression of a coding sequence of interest.

As used herein, “transformed” and “transfected” encompass the introduction of a nucleic acid (e.g., a vector) into a cell by a number of techniques known in the art.

“Plasmids” are designated by a lower case “p” preceded and/or followed by capital letters and/or numbers.

As used herein the term “heterologous” means from another host. The other host can be the same or different species.

The term “plant” is used in it broadest sense. It includes, but is not limited to, any species of woody, ornamental or decorative, crop or cereal, fruit or vegetable plant, and photosynthetic green algae (e.g., Chlamydomonas reinhardtii). It also refers to a plurality of plant cells that is largely differentiated into a structure that is present at any stage of a plant's development. Such structures include, but are not limited to, a fruit, shoot, stem, leaf, flower petal, etc. The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant structure, a plant organ, or a plant tissue.

A “non-naturally occurring plant” refers to a plant that does not occur in nature without human intervention. Non-naturally occurring plants include transgenic plants and plants produced by non-transgenic means such as plant breeding.

The term “plant cell” refers to a structural and physiological unit of a plant, comprising a protoplast and a cell wall. The plant cell may be in the form of an isolated single cell or a cultured cell, or as a part of a higher organized unit such as, for example, a plant tissue, a plant organ, or a whole plant.

The term “plant cell culture” refers to cultures of plant units such as, for example, protoplasts, cell culture cells, cells in plant tissues, pollen, pollen tubes, anthers, ovules, embryo sacs, zygotes and embryos at various stages of development.

The term “plant material” refers to leaves, stems, roots, flowers or flower parts, fruits, pollen, anthers, egg cells, zygotes, seeds, cuttings, cell or tissue cultures, or any other part or product of a plant.

A “plant organ” refers to a distinct and visibly structured and differentiated part of a plant such as a root, stem, leaf, flower bud, inflorescence, seed or embryo.

The term “non-transgenic plant” refers to a plant that has not been genetically engineered to produce polyhydroxyalkanoates or any other recombinant products. A “corresponding non-transgenic plant” refers to the plant prior to the introduction of heterologous nucleic acids that encode enzymes for producing polyhydroxyalkanoates.

### II. Transgenic Plants for Producing PHAs

### A. Representative Plants for Genetic Engineering

It has been discovered that expression of the PHA, for example PHB, biosynthetic pathway and one or more transgenes to increase carbon flow in a plant can yield a plant producing increased levels of PHA including PHB. A preferred plant is switchgrass (Panicum virgatum L.). “Increased levels” refers to amounts of PHA or PHB of more than about 4%, 5%, 6% or 7% dry weight (DW) of plants, for example plants grown in soil rather than plant material from cell culture. In certain embodiments the disclosed transgenic plants produce and accumulate at least about 7% DW PHA. A preferred PHA is poly(3-hydroxybutyrate). The polyhydroxyalkanoates can be homo- or co-polymers.

### 1. C₄ Plants

C₄ plants have a competitive advantage over plants possessing the more common C₃ carbon fixation pathway under conditions of drought, high temperatures and nitrogen limitation. C₄ carbon fixation has evolved on up to 40 independent occasions in different groups of plants, making it an example of convergent evolution. Plants with C₄ metabolism include sugarcane, maize, Sorghum, finger millet, switchgrass, Miscanthus, energy cane, Napier grass, giant reed, and amaranth. C₄ plants represent about 5% of Earth's plant biomass and 1% of its known plant species. However, they account for around 30% of terrestrial carbon fixation. These species are concentrated in the tropics (below latitudes of)45° where the high air temperature contributes to higher possible levels of oxygenase activity by Rubisco, which increases rates of photorespiration in C₃ plants. Suitable C₄ plants include those that do not produce storage materials such as oils and carbohydrates. Representative C₄ plants that can be genetically engineered to produce PHA at significant levels include, but are not limited to, switchgrass, Miscanthus, Sorghum, millets, Napier grass, sugarcane, energy cane, giant reed and other forage and turf grasses.

Additionally, C₄ plants produce lignocellulosic biomass. Lignocellulosic biomass has received considerable attention as an abundant feedstock for biofuels despite the high costs associated with conversion processes. The United States has the agricultural capability to grow vast quantities of this biomass, with recent estimates exceeding one billion tons without affecting food or feed (Perlack et al., (U.S. Department of Energy and U.S. Department of Agriculture) http://feedstockreview.ornl.gov/pdf/billion ton vision.pdf). These estimates include 377 million dry tons of biomass from perennial herbaceous crops that could be dedicated for conversion to biofuels.

A preferred plant to produce PHA is switchgrass, Panicum virgatum L. Switchgrass is a C₄ perennial grass with high biomass yields. It has great potential as an industrial crop in that it requires minimal inputs for growth in many agricultural regions of the United States and Europe (Lewandowski et al., (2003), Biomass Bioenerg, 25:335-361) and has the ability to sequester large amounts of carbon in the soil with its extensive root system (Parrish et al., (2005), Crit Rev Plant Sci, 24:423-459). Direct production of biobased polymers in switchgrass would yield an industrial plant feedstock that could be converted into plastics and fuels, providing better economics for both co-products. Other biomass crops include, but are not limited to, Miscanthus, Sorghum, millets, Napier grass, sugarcane, energy cane, giant reed and other forage and turf grasses.

Both upland and lowland switchgrass cultivars can be used, including but not limited to Alamo, Blackwell, Kanlow, Nebraska 28, Pathfinder, Cave-in-Rock, Shelter and Trailblazer.

### B. Genes for Producing PHB

Genes encoding the enzymes necessary for producing PHA including PHB are known in the art (Madison & Huisman, (1999), Microbiol Mol Biol Rev, 63: 21-53). The PHB biosynthetic pathway requires three enzymatic reactions catalyzed by the following three genes: phaA, phaB, and phaC. The first reaction is the condensation of two acetyl coenzyme A (acetyl-CoA) molecules into acetoacetyl-CoA by β-ketoacyl-CoA thiolase (EC 2.3.1.9) encoded by phaA. The second reaction is the reduction of acetoacetyl-CoA to (R)-3-hydroxybutyryl-CoA by an NADPH-dependent acetoacetyl-CoA reductase (EC 1.1.1.36) encoded by phaB. The (R)-3-hydroxybutyryl-CoA monomers are polymerized into poly(3-hydroxybutyrate) by a PHB synthase encoded by phaC. Sources of these enzymes include, but are not limited to, Zoogloea ramigera, Ralstonia eutropha, Acinetobacter spp., Alcaligenes latus, Pseudomonas acidophila, Paracoccus denitrificans, Rhizobium meliloti, Chromatium vinosum, Thiocystis violacea, and Synechocytis.

In one embodiment, the PHB genes chosen for this construct include a hybrid Pseudomonas oleovorans/Zoogloea ramigera PHA synthase (U.S. Pat. No. 6,316,262 to Huisman et al.) and the thiolase and reductase genes from Ralstonia eutropha (Peoples et al., (1989), J Biol Chem, 264:15293-15297).

### C. Genes to Increase Carbon Flow Towards PHB Synthesis

The levels of sedoheptulose 1,7-bisphosphatase, transketolase, and aldolase enzymes have been shown to have an impact on the control of carbon fixed by the Calvin cycle (Raines, (2003), Photosynth Res, 75:1-10). The FBPase/SBPase gene from Synechococcus elongatus PCC 7942 has previously been expressed in tobacco and enhanced both photosynthesis and plant growth (Miyagawa, (2001), Nat Biotechnol, 19:965-969). Expression of an Arabidopsis SBPase cDNA in tobacco also has resulted in greater plant biomass and increased photosynthetic capacity (Raines, (2003), Photosynth Res, 75:1-10; Lefebvre et al., (2005), Plant Physiol, 138:451-460).

Over-expression of one or more transgenes selected from a bifunctional FBPase/SBPase, an SBPase, an FBPase, a transketolase, or an aldolase with the PHB biosynthetic pathway may increase polymer yield.

Bifunctional enzymes that contain both fructose 1,6-bisphosphatase (EC 3.1.3.11) and sedoheptulose 1,7-bisphosphatase (EC 3.1.3.37) activities have been reported from for example Ralstonia eutropha H16 (Accession number AAA69974), Synechococcus sp. WH 7805 (Accession ZP_{—}01124026), Butyrivibrio crossotus DSM 2876 (Accession number EFF67670), Rothia mucilaginosa DY-18 (Accession number YP_{—}003363264), Thiobacillus denitrificans ATCC 25259 (Accession number AAZ98530), Methylacidiphilum infernorum V4 (Accession number ACD83413), Nitrosomonas europaea ATCC 19718 (Accession number CAD84432), Vibrio vulnificus CMCP6 (Accession number AA009802), Methanohalophilus mahii DSM 5219 (Accession number YP_{—}003542799), and Synechococcus elongatus PCC 7942 (Accession numbers D83512 (SEQ ID NO: 2) and CP000100 (SEQ ID NO: 1)). While the protein encoded by accession number D83512 (SEQ ID NO: 2) has been annotated as FBPase I in the NCBI database, it has been shown to have both FBPase and SBPase activity experimentally (Tamoi et al., (1996), Arch Biochem Biophys, 334:27-36).

Enzymes possessing SBPase activity that could be used to increase the flow of carbon within the Calvin cycle include for example the sedoheptulose-1,7-bisphosphatase from Zea mays (Accession NP_{—}001148402), the sedoheptulose-1,7-bisphosphatase from Arabidopsis thaliana (Accession

AAB33001), the sedoheptulose-1,7-bisphosphatase from Triticum aestivum (Accession P46285), or the redox-independent sedoheptulose-1,7-bisphosphatase from Chlamydomonas reinhardtii (Accession No. XM_{—}001691945).

Enzymes possessing FBPase that could be used to increase the flow of carbon within the Calvin cycle include for example the protein encoded by the fbpI gene from Synechococcus elongatus PCC 6301 (Accession number AP008231.1), the gene encoding fructose-1,6-bisphosphatase from Zea mays (Accession NP_{—}001147459), the gene encoding fructose-1, 6-bisphosphatase from Saccharum hybrid cultivar H65-7052 (Accession CAA61409), the gene encoding fructose-1,6-bisphosphatase from Pisum sativum (Accession AAD10213) or the recently identified redox-independent FBPaseII gene from Fragaria×ananassa (Accession No. EU185334).

### III. Plant Transformation Technology

A. Transformation of Plants with PHA Genes

Transgenic plants for producing PHA, in particular PHB, can be produced using conventional techniques to express phaA, phaB, and phaC in plants or plant cells (Methods in Molecular Biology, vol. 286, Transgenic Plants: Methods and Protocols Edited by L. Pena, Humana Press, Inc. Totowa, N.J. (2005)). Typically, gene transfer, or transformation, is carried out using explants capable of regeneration to produce complete, fertile plants. Generally, a DNA or an RNA molecule to be introduced into the organism is part of a transformation vector. A large number of such vector systems known in the art may be used, such as plasmids. The components of the expression system can be modified, e.g., to increase expression of the introduced nucleic acids. For example, truncated sequences, nucleotide substitutions or other modifications may be employed. Expression systems known in the art may be used to transform virtually any plant cell under suitable conditions. A transgene comprising a DNA molecule encoding the genes for PHA production is preferably stably transformed and integrated into the genome of the host cells. Transformed cells are preferably regenerated into whole plants. Detailed description of transformation techniques are within the knowledge of those skilled in the art.

### B. Reporter Genes and Selectable Marker Genes

Reporter genes or selectable marker genes may be included in the expression cassette. Examples of suitable reporter genes known in the art can be found in, for example, Jefferson et al. (1991) in Plant Molecular Biology Manual, ed. Gelvin et al. (Kluwer Academic Publishers), pp. 1-33; DeWet et al., (1987), Mol Cell Biol 7:725-737; Goff et al., (1990), EMBO J, 9:2517-2522; Kain et al., (1995), Bio Techniques, 19:650-655; and Chiu et al., (1996), Current Biology, 6:325-330.

Selectable marker genes for selection of transformed cells or tissues and plants obtained from them can include genes that confer antibiotic resistance or resistance to herbicides. Examples of suitable selectable marker genes include, but are not limited to, genes encoding resistance to chloramphenicol (Herrera Estrella et al., (1983), EMBO J, 2:987-992), methotrexate (Herrera Estrella et al., (1983), Nature, 303:209-213; Meijer et al, (1991), Plant Mol Biol, 16:807-820); hygromycin (Waldron et al., (1985), Plant Mol Biol, 5:103-108; Zhijian et al., (1995), Plant Sci, 108:219-227); streptomycin (Jones et al., (1987), Mol Gen Genet, 210:86-91); spectinomycin (Bretagne-Sagnard et al., (1996), Transgenic Res, 5:131-137); bleomycin (Hille et al., (1990), Plant Mol Biol, 7:171-176) ; sulfonamide (Guerineau et al., (1990), Plant Mol Biol, 15:127-136); bromoxynil (Stalker et al., (1988), Science, 242:419-423); glyphosate (Shaw et al., (1986), Science, 233:478-481); phosphinothricin (DeBlock et al., (1987), EMBO J, 6:2513-2518).

Other genes that could be useful in the recovery of transgenic events but might not be required in the final product include, but are not limited to, GUS (β-glucoronidase (Jefferson, (1987), Plant Mol Biol Rep, 5:387), GFP (green fluorescent protein) (Chalfie et al., (1994), Science, 263:802), luciferase (Riggs et al., (1987), Nucleic Acids Res, 15:8115; Luehrsen et al., (1992), Methods Enzymol, 216:397-414) and the maize genes encoding for anthocyanin production (Ludwig et al., (1990), Science, 247:449).

The expression cassette including a promoter sequence operably linked to a heterologous nucleotide sequence of interest, for example encoding a PHA synthase, a thiolase, and/or a reductase can be used to transform any plant.

### C. Transformation Protocols

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al., (1986), Biotechniques, 4:320-334), electroporation (Riggs et al., (1986), Proc Natl Acad Sci USA, 83:5602-5606), Agrobacterium-mediated transformation (U.S. Pat. No. 5,563,055 to Townsend et al.; WO U.S. 98/01268 to Zhao et al.) and direct gene transfer (Paszkowski et al., (1984), EMBO J, 3:2717-2722) by microprojectile bombardment (see, for example, U.S. Pat. No. 4,945,050 to Sanford et al.; Tomes et al., (1995), Plant Cell, Tissue, and Organ Culture: Fundamental Methods, ed. Gamborg and Phillips (Springer-Verlag, Berlin); and McCabe et al., (1988), Biotechnology, 6:923-926). Also see Weissinger et al., (1988), Ann Rev Genet, 22:421-477 and Sanford et al., (1987), Particulate Science and Technology, 5:27-37 (onion); Christou et al., (1988), Plant Physiol, 87:671-674, McCabe et al,. (1988), BioTechnology, 6:923-926, Finer & McMullen, (1991), In Vitro Cell Dev Biol, 27P:175-182, and Singh et al., (1998), Theon Appl Genet, 96:319-324 (soybean); Dafta et al., (1990), Biotechnology, 8:736-740 (rice); Klein et al., (1988), Proc Natl Acad Sci USA, 85:4305-4309, U.S. Pat. No. 5,240,855 to Tomes, U.S. Pat. Nos. 5,322,783 and 5,324,646 to Buising et al., Tomes et al. (1995) in Plant Cell, Tissue, and Organ Culture: Fundamental Methods, ed. Gamborg (Springer-Verlag, Berlin), Klein et al., (1988), Plant Physiol, 91:440-444, and Fromm et al,. (1990), Biotechnology, 8:833-839 (maize); Hooykaas-Van Slogteren et al., (1984), Nature, 311:763-764 and U.S. Pat. No. 5,736,369 to Bowen et al. (cereals); Bytebier et al., (1987), Proc Natl Acad Sci USA, 84:5345-5349 (Liliaceae); De Wet et al., (1985), in The Experimental Manipulation of Ovule Tissues, ed. Chapman et al. (Longman, N.Y.), pp. 197-209 (pollen); Kaeppler et al,. (1990), Plant Cell Rep, 9:415-418 and Kaeppler et al., (1992), Theor Appl Genet, 84:560-566 (whisker-mediated transformation); D'Halluin et al., (1992), Plant Cell, 4:1495-1505 (electroporation); Li et al,. (1993), Plant Cell Rep, 12:250-255 and Christou & Ford, (1995), Ann Bot, 75:407-413 (rice); Osjoda et al., (1996), Nat Biotechnol, 14:745-750 (maize via Agrobacterium tumefaciens).

The transformed cells are grown into plants in accordance with conventional techniques. See, for example, McCormick et al., (1986), Plant Cell Rep, 5:81-84. These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

### D. Modulating Expression of Genes in Plants

### 1. Inducible Promoters

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize 1n2-2 promoter, which is activated by benzenesulfonamide herbicide safeners, the maize GST promoter, which is activated by hydrophobic electrophilic compounds that are used as pre-emergent herbicides, and the tobacco PR-1 promoter, which is activated by salicylic acid. Other chemical-regulated promoters include steroid-responsive promoters (see, for example, the glucocorticoid-inducible promoter (Schena et al., (1991), Proc Natl Acad Sci USA, 88:10421-10425; McNellis et al., (1998), Plant J, 14:247-257) and tetracycline-inducible and tetracycline-repressible promoters (see, for example, Gatz et al., (1991), Mol Gen Genet, 227:229-237; U.S. Pat. Nos. 5,814,618 and 5,789,156, herein incorporated by reference in their entirety).

In one embodiment, coordinated expression of the three transgenes, phaA, phaB, and phaC, necessary for conversion of acetyl-CoA to PHB, is controlled by the maize light inducible cab-m5 promoter in multi-gene expression constructs (Sullivan et al., (1989), Mol Gen Genet, 215:431-440; Becker et al., (1992), Plant Mol Biol, 20:49-60). The promoter can be fused to the hsp70 intron (U.S. Pat. No. 5,593,874 to Brown et al.) for enhanced expression in monocots. It has been previously shown that plants transformed with multi-gene constructs produced higher levels of polymer than plants obtained from crossing single transgene lines (Bohmert et al., (2000), Planta, 211:841-845; Valentin et al., (1999), Int J Biol Macromol, 25:303-306).

### 2. Constitutive Promoters

Constitutive promoters include, for example, the core promoter of the Rsyn7 promoter and other constitutive promoters disclosed in WO 99/43838 and U.S. Pat. No. 6,072,050, the core CAMV 35S promoter (Odell et al., (1985), Nature, 313:810-812), rice actin (McElroy et al., (1990), Plant Cell, 2:163-171), ubiquitin (Christensen et al., (1989), Plant Mol Biol, 12:619-632; Christensen et al., (1992), Plant Mol Biol, 18:675-689), pEMU (Last et al., (1991), Theor Appl Genet, 81:581-588), MAS (Velten et al., (1984), EMBO J, 3:2723-2730), ALS promoter (U.S. Pat. No. 5,659,026). Other constitutive promoters are described in U.S. Pat. Nos. 5,608,149; 5,608,144; 5,604,121; 5,569,597; 5,466,785; 5,399,680; 5,268,463; and 5,608,142.

In one embodiment, coordinated expression of the three transgenes, phaA, phaB, and phaC, necessary for conversion of acetyl-CoA to PHB is controlled by the constitutive rice ubiquitin 2 promoter in multi-gene expression constructs.

Preferred promoters include, but are not limited to, constitutive rice ubiquitin 2 or the maize light inducible cab-m5 promoter.

### 3. Weak Promoters

Where low level expression is desired, weak promoters may be used. Generally, the term “weak promoter” is intended to describe a promoter that drives expression of a coding sequence at a low level. “Low level” refers to levels of about 1/1000 transcripts to about 1/100,000 transcripts to about 1/500,000 transcripts. Alternatively, it is recognized that weak promoters also encompasses promoters that are expressed in only a few cells and not in others to give a total low level of expression. Where a promoter is expressed at unacceptably high levels, portions of the promoter sequence can be deleted or modified to decrease expression levels.

Such weak constitutive promoters include, for example, the core promoter of the Rsyn7 promoter (WO 99/43838 and U.S. Pat. No. 6,072,050).

### 4. Tissue Specific Promoters

“Tissue-preferred” promoters can be used to target gene expression within a particular tissue. Tissue-preferred promoters include those described by Yamamoto et al., (1997), Plant J, 12:255-265, Kawamata et al., (1997), Plant Cell Physiol, 38:792-803, Hansen et al., (1997), Mol Gen Genet, 254:337-343, Russell et al., (1997), Transgenic Res, 6:157-168, Rinehart et al., (1996), Plant Physiol, 112:1331-1341, Van Camp et al., (1996), Plant Physiol, 112:525-535, Canevascini et al., (1996), Plant Physiol, 112:513-524, Yamamoto et al., (1994), Plant Cell Physiol, 35:773-778, Lam, (1994), Results Probl Cell Differ, 20:181-196, Orozco et al., (1993), Plant Mol Biol, 23:1129-1138, Matsuoka et al., (1993), Proc Natl Acad Sci USA, 90:9586-9590, and Guevara-Garcia et al., (1993), Plant J, 4:495-505. Such promoters can be modified, if necessary, for weak expression.

i. Seed Specific Promoters

“Seed-preferred” promoters include both “seed-specific” promoters (those promoters active during seed development such as promoters of seed storage proteins) as well as “seed-germinating” promoters (those promoters active during seed germination). See Thompson et al., (1989), BioEssays 10:108, herein incorporated by reference. Such seed-preferred promoters include, but are not limited to, Ciml (cytokinin-induced message); cZ19B1 (maize 19 kDa zein); milps (myo-inositol-1-phosphate synthase); and ce1A (cellulose synthase). Gamma-zein is a preferred endosperm-specific promoter. Glob-1 is a preferred embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, and globulin 1.

ii. Leaf Specific Promoters

Leaf-specific promoters are known in the art. See, for example, Yamamoto et al., (1997), Plant J, 12:255-265, Kwon et al., (1994), Plant Physiol, 105:357-67, Yamamoto et al., (1994), Plant Cell Physiol, 35:773-778, Gotor et al., (1993), Plant J, 3:509-518, Orozco et al., (1993), Plant Mol Biol, 23:1129-1138, and Matsuoka et al., (1993), Proc Natl Acad Sci USA, 90:9586-9590.

iii. Root Specific Promoters

Root-preferred promoters are known and may be selected from the many available from the literature or isolated de novo from various compatible species. See, for example, Hire et al., (1992), Plant Mol Biol, 20:207-218 (soybean root-specific glutamine synthetase gene), Keller & Baumgartner, (1991), Plant Cell, 3:1051-1061 (root-specific control element in the GRP 1.8 gene of French bean); Sanger et al., (1990), Plant Mol Biol, 14:433-443 (root-specific promoter of the mannopine synthase (MAS) gene of Agrobacterium tumefaciens), and Miao et al., (1991), Plant Cell, 3:11-22 (full-length cDNA clone encoding cytosolic glutamine synthetase (GS), which is expressed in roots and root nodules of soybean). See also U.S. Pat. Nos. 5,837,876; 5,750,386; 5,633,363; 5,459,252; 5,401,836; 5,110,732; and 5,023,179.

### 5. Combinations of Promoters

Certain embodiments use transgenic plants or plant cells having multi-gene expression constructs harboring more than one promoter. The promoters can be the same or different.

### E. Chloroplast Targeting Sequences

In one embodiment, the chloroplast was chosen as the site for PHB synthesis in switchgrass since this organelle has an endogenous flux of the polymer precursor acetyl-CoA for fatty acid biosynthesis and has yielded the highest levels of polymer in plants to date (Bohmert et al., (2004), Molecular Biology and Biotechnology of Plant Organelles (Daniell H and Chase CD eds): 559-585, Netherlands: Kluwer Academic Publishers).

Chloroplast targeting sequences are known in the art and can be found at the N-terminus of proteins including the small subunit of ribulose-1,5-bisphosphate carboxylase (Rubisco) (de Castro Silva Filho et al., (1996), Plant Mol Biol, 30:769-780; Schnell et al., (1991), J Biol Chem, 266:3335-3342), 5-(enolpyruvyl)shikimate-3-phosphate synthase (EPSPS) (Archer et al., (1990), J Bioenerg Biomemb, 22:789-810); tryptophan synthase (Zhao et al., (1995), J Biol Chem, 270:6081-6087); plastocyanin (Lawrence et al., (1997), J Biol Chem, 272:20357-20363); chorismate synthase (Schmidt et al., (1993), J Biol Chem, 268:27447-27457), and the light harvesting chlorophyll a/b binding protein (LHBP) (Lamppa et al., (1988), J Biol Chem, 263:14996-14999). See also Von Heijne et al., (1991), Plant Mol Biol Rep, 9:104-126, Clark et al., (1989), J Biol Chem, 264:17544-17550, Della-Cioppa et al., (1987), Plant Physiol, 84:965-968, Romer et al., (1993), Biochem Biophys Res Commun, 196:1414-1421, and Shah et al., (1986), Science, 233:478-481.

### F. Methods for Transforming Chloroplasts

An alternative method for engineering PHB production in plants is direct integration of the genes of interest into the chloroplast genome.

Methods for transformation of chloroplasts are known in the art. See, for example, Svab et al., (1990), Proc Natl Acad Sci USA, 87:8526-8530, Svab & Maliga, (1993), Proc Natl Acad Sci USA, 90:913-917, and Svab & Maliga, (1993), EMBO J, 12:601-606. The method relies on particle gun delivery of DNA containing a selectable marker and targeting of the DNA to the plastid genome through homologous recombination. Additionally, plastid transformation may be accomplished by transactivation of a silent plastid-born transgene by tissue-preferred expression of a nuclear-encoded and plastid-directed RNA polymerase. Such a system was reported in McBride et al., (1994), Proc Natl Acad Sci USA, 91:7301-7305.

Plastid transformation technology is extensively described in U.S. Pat. Nos. 5,451,513; 5,545,817; and 5,545,818, in WO 95/16783, and in McBride et al., (1994), Proc Natl Acad Sci USA, 91:7301-7305. A basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al., (1990), Proc Natl Acad Sci USA, 87:8526-8530; Staub & Maliga, (1992), Plant Cell, 4:39-45). The presence of cloning sites between these markers allows creation of a plastid targeting vector for introduction of foreign DNA molecules (Svab & Maliga, (1993), EMBO J, 12:601-606). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab & Maliga, (1993), Proc Natl Acad Sci USA, 90:913-917). This marker has been used successfully for high-frequency transformation of the plastid genome of the green alga Chlamydomonas reinhardtii (Goldschmidt-Clermont, (1991), Nucl Acids Res, 19:4083-4089). Other selectable markers useful for plastid transformation are known in the art.

The nucleic acids of interest to be targeted to the chloroplast may be optimized for expression in the chloroplast to account for differences in codon usage between the plant nucleus and this organelle. Modification of the gene encoding sequence to contain chloroplast-preferred codons is described in U.S. Pat. No. 5,380,831.

An alternative method for plastid transformation as described in WO 2010/061186 wherein RNA produced in the nucleus of a plant cell can be targeted to the plastids and integrated into the plastome can also be used to practice the disclosed methods and compositions.

### G. Requirements for Construction of Plant Expression Cassettes

Nucleic acid sequences intended for expression in transgenic plants are first assembled in expression cassettes behind a suitable promoter active in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be transferred to the plant transformation vectors described infra. The following is a description of various components of typical expression cassettes.
1. Transcriptional Terminators

A variety of transcriptional terminators are available for use in expression cassettes. These are responsible for the termination of transcription beyond the transgene and the correct polyadenylation of the transcripts.

Appropriate transcriptional terminators are those that are known to function in plants and include the CaMV 35S terminator, the tml terminator, the nopaline synthase terminator and the pea rbcS E9 terminator. These are used in both monocotyledonous and dicotyledonous plants.
2. Sequences for the Enhancement or Regulation of Expression

Numerous sequences have been found to enhance gene expression from within the transcriptional unit and these sequences can be used in conjunction with the genes to increase their expression in transgenic plants. For example, various intron sequences such as introns of the maize Adhl gene have been shown to enhance expression, particularly in monocotyledonous cells. In addition, a number of non-translated leader sequences derived from viruses are also known to enhance expression, and these are particularly effective in dicotyledonous cells.

### H. Coding Sequence Optimization

The coding sequence of the selected gene may be genetically engineered by altering the coding sequence for optimal expression in the crop species of interest. Methods for modifying coding sequences to achieve optimal expression in a particular crop species are well known (see, e.g., Perlak et al., (1991), Proc Natl Acad Sci USA, 88:3324 and Koziel et al., (1993), Biotechnology, 11:194).

### I. Construction of Plant Transformation Vectors

Numerous transformation vectors available for plant transformation are known to those of ordinary skill in the plant transformation arts. The genes pertinent to this disclosure can be used in conjunction with any such vectors. The selection of vector depends upon the selected transformation technique and the target species for transformation. For certain target species, different antibiotic or herbicide selection markers are preferred. Selection markers used routinely in transformation include the nptII gene, which confers resistance to kanamycin and related antibiotics (Messing & Vierra, (1982), Gene, 19:259-268; Bevan et al., (1983), Nature, 304:184-187), the bar gene, which confers resistance to the herbicide phosphinothricin (White et al., (1990), Nucl Acids Res, 18:1062; Spencer et al., (1990), Theor Appl Genet, 79:625-631), the hptII gene, which confers resistance to the antibiotic hygromycin (Blochinger & Diggelmann, Mol. Cell Biol., 4: 2929-2931), the manA gene, which allows for positive selection in the presence of mannose (Miles & Guest, (1984), Gene, 32:41-48; U.S. Pat. No. 5,767,378), the dhfr gene, which confers resistance to methotrexate (Bourouis et al., (1983), EMBO J, 2:1099-1104), and the EPSPS gene, which confers resistance to glyphosate (U.S. Pat. Nos. 4,940,935 and 5,188,642).

Many vectors are available for transformation using Agrobacterium tumefaciens. These typically carry at least one T-DNA sequence and include vectors such as pBIN19. Typical vectors suitable for Agrobacterium transformation include the binary vectors pCIB200 and pCIB2001, as well as the binary vector pCIB 10 and hygromycin selection derivatives thereof. (See, for example, U.S. Pat. No. 5,639,949).

Transformation without the use of Agrobacterium tumefaciens circumvents the requirement for T-DNA sequences in the chosen transformation vector and consequently vectors lacking these sequences are utilized in addition to vectors such as the ones described above which contain T-DNA sequences. Transformation techniques that do not rely on Agrobacterium include transformation via particle bombardment, protoplast uptake (e.g., PEG and electroporation) and microinjection. The choice of vector depends largely on the preferred selection for the species being transformed. Typical vectors suitable for non-Agrobacterium transformation include pCIB3064, pSOG 19, and pSOG35 (see, for example, U.S. Pat. No. 5,639,949).

J. Prescreening of Cultures from Different Genotypes

One embodiment provides a method for increasing the efficiency of transforming plant tissue by preselecting the plant material. For example, mature caryopses can be induced to form highly embryogenic callus cultures (Denchev & Conger, (1994), Crop Sci, 34:1623-1627). Dedifferentiation of caryopses into embryogenic callus cultures can be achieved using numerous basal media with various plant growth hormones. Callus induction from caryopses, young leaf tissue, portions of seedlings, and immature inflorescences can be achieved using a cytokinin in the growth medium. In one embodiment, production of embryogenic calluses can be obtained in the presence of 2,4-dichlorophenoxyacetic acid (2,4-D) and/or 6-benzylaminopurine (BAP). After multiple transfers onto a fresh medium for callus growth, the regeneration potential of these embryogenic callus cultures is evaluated. Cultures capable of producing about 300 or more plantlets per gram of callus are further propagated and pooled for transformation. Alternatively cultures capable of producing about 200 or more plantlets can be used. The cultures are then transformed using conventional techniques, preferably incubation with Agrobacterium.

### K. Transformation and Selection of Callus Cultures and Plants

The embryogenic cultures are infected and co-cultivated with an Agrobacterium strain carrying the gene constructs encoding enzymes for PHA production with a selectable marker and/or reporter gene. In one embodiment, genes for the production of PHB (phaA, phaB, and phaC) are used. In another embodiment, Agrobacterium tumefaciens strain AGL1 is used. In an alternative embodiment, infection and co-cultivation is performed in the presence of acetosyringone. The cultures can then be selected using one or more of the selection methods described above which are well known to those skilled in the art. In a preferred embodiment, selection occurs by incubating the cultures on a callus growth medium containing bialaphos. In an alternative embodiment, selection can occur in the presence of hygromycin. Resistant calluses are then cultured on a regeneration medium (Somleva, 2006, Agrobacterium Protocols Wang K., ed, pp 65-74: Humana Press; Somleva et al., (2002), Crop Sci, 42:2080-2087) containing the preferred selection agent.

L. Initiation and Re-transformation of Cultures from Transgenic Plants

In one embodiment, stably transformed plants are used as a source of explants for culture initiation and plant regeneration. In a preferred embodiment, in vitro developed panicles are obtained from the top culm node of elongating tillers from switchgrass plants engineered for the production of PHB (WO 2010102220 A1; U.S. 2010/0220256 A1). The starting material can be obtained from primary transformants, plants propagated from them through immature inflorescence-derived callus cultures or nodal segments, or plants grown from seeds obtained from controlled crosses between transgenic plants or between transgenic and non-transgenic, wild-type plants.

For callus initiation, individual spikelets from panicles formed in tissue culture are plated on MS medium for callus initiation and growth (Denchev & Conger, (1994), Crop Sci, 34:1623-1627). Resultant embryogenic callus cultures are incubated at 28° C., in the dark and propagated by monthly transfers on to a fresh medium (Somleva, 2006, Agrobacterium Protocols Wang K., ed, pp 65-74: Humana Press). Plants can be obtained by transferring callus pieces on MS medium for plant regeneration (Denchev & Conger, (1994), Crop Sci, 34:1623-1627) and incubating them in the light (Somleva, 2006, Agrobacterium Protocols Wang K., ed, pp 65-74: Humana Press). All of the regenerated plants are transgenic and produce polymer as demonstrated previously (WO 2010102220 A1; U.S. 2010/0220256 A1).

The immature inflorescence-derived callus cultures from transgenic plants can also be used as a target material for introduction of additional recombinant genes into transgenic lines with desired characteristics. This approach could be used for engineering of new metabolic pathways, for manipulations of the metabolite flux through competing and interconnected pathways, and for improvement of various agronomic traits.

### IV. Methods of Use

The disclosed transgenic plants can be used to produce PHAs, in particular poly(3-hydroxybutyrate), as well as lignocellulosic biomass. Plants are typically produced by seeding of prepared fields, then harvesting the biomass using conventional hay or grain harvesting equipment. Polymer is extracted by solvent extraction in most cases, and then processed using standard techniques.

The PHB can be used in a variety of applications including packaging products like bottles, bags, wrapping film and other biodegradable devices. PHB may have medical device applications due to its biodegradability, optical activity and isotacticity. Alternatively the PHA can be recovered from the biomass in the form of a chemical intermediate by appropriate treatment of the biomass using catalytic or thermal methods.

The lignocellulosic biomass materials can be used to produce biofuels via cellulose hydrolysis, production of pyrolysis liquids or syngas, and/or cogeneration of power and steam (Snell & Peoples, (2009), Biofuels Bioprod Bioref 3:456-467). By making use of all of the plant material additional value is obtained.

Thus, one embodiment provides plant feedstock or plant material including at least about 3% to about 7% polyhydroxyalkanoate, preferably poly(3-hydroxybutyrate), and lignocellulosic biomass, wherein the plant does not produce storage products such as oils or carbohydrates. Preferably the plant is switchgrass. The PHA and the lignocellulosic biomass can be extracted from the feedstock using conventional methods.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

Design and Construction of Transformation Vectors Expressing a Gene Encoding FBPase/SBPase with Genes Encoding the PHB Biosynthetic Enzymes in Switchgrass.

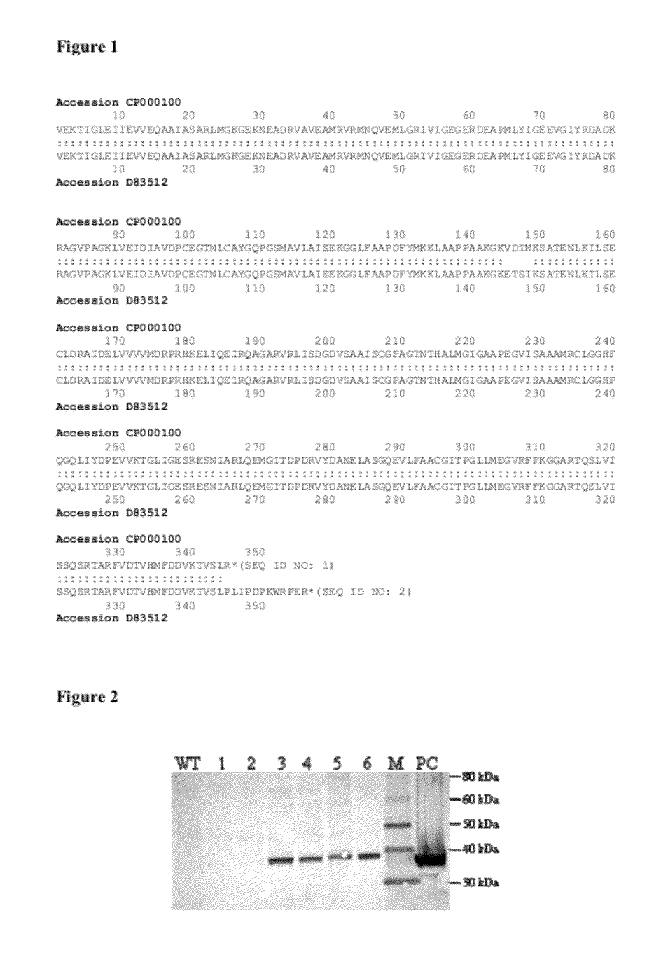

The effect of expressing a gene encoding FBPase/SBPase from Synechococcus elongatus PCC 7942 was examined in both wild-type and PHB producing switchgrass plants. Two different sequences for the FBPase/SBPase from Synechococcus elongatus PCC 7942 are listed in the NCBI database, accession numbers D83512 (SEQ ID NO: 2) and CP000100 (SEQ ID NO: 1). These two sequences are 95% identical and differ at amino acids 145 to 148 and at their C-terminus (FIG. 1). The gene listed in Accession # CP000100 (SEQ ID

NO: 1) is annotated as an FBPase/SBPase in the data base whereas the gene listed in Accession # D83512 (SEQ ID NO: 2) is annotated as FBPase I. Despite its annotation as FBPase I, accession D83512 (SEQ ID NO: 2) has been shown to encode a bi-functional enzyme with both FBPase and SBPase activities using in vitro enzyme assays (Tamoi et al., (1996), Arch Biochem Biophys, 334:27-36) and has previously been shown to enhance photosynthesis and plant growth in tobacco (Miyagawa, (2001), Nat Biotechnol, 19:965-969). A gene was isolated by PCR from genomic DNA prepared from Synechococcus elongatus PCC 7942 (Synechococcus elongatus ATCC 33912) using primers KMB 9 (5′—CC gAA TTC gTg gAg AAg ACg ATC ggT CTC g—3′ (SEQ ID NO: 5)) and KMB 10 (5′—CC TCT AgA CTA CCg CTC Cgg CCg CCA TTT g—3′ (SEQ ID NO: 6)). Sequencing of PCR products yielded a DNA sequence 100% identical to accession number CP000100 (SEQ ID NO: 1).

The gene encoding the FBPase/SBPase from accession number CP000100 (SEQ ID NO: 1) was verified to encode an active protein by measuring FBPase activity. The FBPase/SBPase gene was cloned into the E. coli expression vector pSE380 forming plasmid pMBXS364 and transformed into E. coli. Enzyme assays of FBPase activity were performed essentially as described by Tamoi et al. (1996). In a final volume of 1 mL, the reaction mixture for FBPase assays contained 200 mM Tris-HCl, pH 8.0, 10 mM MgCl₂, 0.5 mM EDTA, 0.4 mM NADP⁺, 0.1 mM D-fructose-1,6-bisphosphate, 1 Unit D-glucose-6-phosphate dehydrogenase, and 3 Units of phosphoglucoisomerase. The reactions were initiated by the addition of crude soluble extract. The reactions were carried out at 25° C. and the formation of NADPH was monitored at 340 nm for 10 min. Protein concentrations were determined using the Bradford assay with a BSA standard curve. Crude extracts of E. coli cells containing the FBPase/SBPase expression vector possessed 0.18 Units/mg of activity where one Unit is defined as the amount of enzyme that hydrolyzes one μmol of substrate per minute. Control E. coli extracts that did not contain plasmid pMBXS364 expressing the FBPase/SBPase gene possessed 0.0014 U/mg of activity.

Plant transformation vector pMBXS422 (SEQ ID NO: 3) for transformation of switchgrass was prepared. It contains the vector backbone from pCAMBIA1330 with an expression cassette for plastid targeted FBPase/SBPase. The coding sequence for FBPase/SBPase is fused to a DNA fragment encoding the signal peptide of the small subunit of Rubisco from pea (Pisum sativum) and the first 24 amino acids of the mature protein (Cashmore, (1983), In Genetic Engineering of Plants: An Agricultural Perspective (Kosuge, T., Meredith, C. P. and Hollaender, A., eds), pp. 29-38. New York: Plenum Publications Corp.), allowing targeting of the protein to the chloroplasts. The expression of the transgenes is under the control of the cab-m5 light-inducible promoter of the chlorophyll a/b-binding protein in maize (Sullivan et al., (1989), Mol Gen Genet, 215:431-440; Becker et al., (1992), Plant Mol Biol, 20:49-60) fused to the heat shock protein 70 (hsp70) intron (U. S. Pat. No. 5,593,874). This binary vector also possesses an expression cassette for the selectable marker gene hptll, conferring resistance to hygromycin, whose expression is controlled by the CaMV35S promoter.

An additional transformation vector, named pMBXS424 (SEQ ID NO: 7), for co-expression of the FBPase/SBPase gene with the PHB biosynthetic enzymes was also prepared using the previously described pMBXS 155 as a starting vector (Somleva et al., (2008), Plant Biotechnol J, 6:663-678). The plant transformation vector pMBXS 155 contains the following expression cassettes: (1) an expression cassette for PHA synthase containing the cab-m5 promoter fused to the heat shock protein 70 intron (cab-m5/hsp70), a DNA fragment encoding the signal peptide of the small subunit of Rubisco from pea (P. sativum) and the first 24 amino acids of the mature protein, a DNA fragment encoding a hybrid PHA synthase (PhaC; U. S. Pat. No. 6,316,262) in which the first nine amino acids at the N-terminus of this synthase are derived from the Pseudomonas oleovorans phaC1 gene and the remainder of the synthase coding sequence is derived from Zoogloea ramigera phaC gene, and a polyadenylation sequence [3′ termination sequence of nopaline synthase (nos)]; (2) an expression cassette for reductase containing the cab-m5/hsp70 promoter fragment, a DNA fragment encoding the signal peptide and the first 24 amino acids of the mature protein of the small subunit of Rubisco from pea, a DNA fragment encoding a NADPH dependent reductase (PhaB) from Ralstonia eutropha (Peoples & Sinskey, (1989), J Biol Chem, 264:15293-15297), and nos; (3) an expression cassette for thiolase containing cab-m5/hsp70 promoter fragment, a DNA fragment encoding the signal peptide and the first 24 amino acids of the mature protein of the small subunit of Rubisco from pea, the phaA gene encoding a β-ketothiolase (PhaA) from Ralstonia eutropha (Peoples & Sinskey, (1989), J. Biol Chem, 264:15293-15297), and nos; (4) an expression cassette for selection of transformants consisting of the double enhanced version of the 35S promoter from cauliflower mosaic virus (CaMV) fused to the hsp70 intron, a bar gene encoding phosphinothricin acetyltransferase imparting resistance to bialaphos, and a polyadenylation sequence.

Insertion of the expression cassette for plastid targeted FBPase/SBPase described in pMBXS422 (SEQ ID NO: 3) into pMBXS155 yielded pMBXS424 (SEQ ID NO: 7).

TABLE 1
Summary of plant transformation vectors for expression of
FBPase/SBPase

| | | Selectable |
|---|---|---|
| Vector | Genes | Marker |
| pMBXS422 | FBPase/SBPase | hptII |
| (SEQ ID NO: 3) | | |
| pMBXS155 | phaA, phaB, phaC | bar |
| pMBXS424 | FBPase/SBPase, phaA, | bar |
| (SEQ ID NO: 7) | phaB, phaC | |

### Example 2

Re-Transformation of PHB Producing Switchgrass Lines with the Synechococcus PCC 7942 FBP/SBPase Genes.

Transgenic switchgrass plants carrying the PHB pathway genes under the control of the maize cab-m5 promoter (Somleva et al., (2008), Plant Biotechol J, 6:663-678; U.S. 2009/0271889 A1) were used for initiation of immature inflorescence-derived callus cultures. These donor plants were obtained from immature inflorescence-derived cultures initiated either from polymer producing primary transformants or from plants micropropagated from them through inflorescence-derived callus cultures (WO 2010102220 A1; U.S. 2010/0220256 A1). Five of the lines used in these experiments were obtained from the well characterized primary transformant 56-2a-1/3 (Somleva et al., (2008), Plant Biotechol J, 6:663-678) and one line was derived from another T₀ plant from the same genotype. These PHB producing switchgrass plants were grown under greenhouse conditions and the top culm nodes of elongating tillers (3-4 visible nodes) were used for production of inflorescences in tissue culture following the previously published procedure for non-transformed switchgrass plants (Alexandrova et al., (1996), Crop Sci, 36:175-178). Callus cultures were initiated from individual spikelets from in vitro developed panicles and propagated by transferring on to a fresh medium for callus growth (Denchev & Conger, (1994), Crop Sci, 34:1623-1627) every four weeks as described previously (WO 2010102220 A1; U.S. 2010/0220256 A1). Immature inflorescence-derived callus cultures initiated from different donor plants were maintained for up to 6 months at 27° C., in the dark. For plant regeneration, calluses were plated on MS medium supplemented with 1.4 μM gibberellic acid and incubated at 27° C. with a 16-h photoperiod (cool white fluorescent bulbs, 80 μmol/m²/s) for four weeks followed by a transfer on to a fresh regeneration medium for another four weeks. As reported previously, callus cultures initiated from in vitro developed panicles from both wild-type and PHB producing switchgrass plants possess high embryogenic and regeneration potential (WO 2010102220 A1;U.S. 2010/0220256 A1). Cultures from the six lines used in this study formed 833-1246 plantlets per gram callus prior to re-transformation. The highly embryogenic immature inflorescence-derived callus cultures initiated from the six PHB producing switchgrass lines were transformed with Agrobacterium tumefaciens carrying pMBXS422 (SEQ ID NO: 3), following previously published protocols for transformation of mature caryopsis-derived switchgrass callus cultures (Somleva et al., (2002), Crop Sci, 42:2080-2087; Somleva, (2006), Agrobacterium Protocols, Wang K., ed., pp 65-74, Humana Press). In total, 578 callus pieces were inoculated with Agrobacterium tumefaciens strain AGL1 carrying pMBXS422 (SEQ ID NO: 3). Transformed cultures were selected with 200 mg/L hygromycin as described elsewhere (WO 2010102220 A1; U.S. 2010/0220256 A1). The identified 186 hygromycin-resistant calluses produced 2,352 re-transformed plantlets after selection with the antibiotic for 4-6 weeks. The presence of the FBPase/SBPase transgenes was confirmed by PCR.

Portions from the immature inflorescence-derived callus cultures initiated from the six PHB producing lines used in these experiments were plated on plant regeneration medium prior to re-transformation. Plants obtained from these cultures were used as controls in analyses of PHB production, photosynthetic activity, plant growth rate and biomass accumulation in tissue culture and soil.

Immature inflorescence-derived callus cultures initiated from non-transformed, wild-type plants from the same Alamo genotype were plated on a regeneration medium and the resultant plantlets were grown under the same in vitro and greenhouse conditions. These wild-type plants served as controls for photosynthetic activity measurements, plant growth rate and biomass accumulation in tissue culture and soil.

Leaf tissues (10-20 mg) from primary transformants in tissue culture were collected, lyophilized and prepared for analysis by gas chromatography/mass spectroscopy (GC/MS) using a previously described simultaneous extraction and butanolysis procedure (Kourtz et al., (2007), Transgenic Res, 16:759-769). In tissue culture, polymer content was measured in more than 220 re-transformed plants and 100 control PHB producing plants prior to transfer to soil. No significant differences were detected in the PHB levels in plantlets re-transformed with pMBXS422 (SEQ ID NO: 3) compared to the control plants.

However, differences in PHB production were determined in re-transformed and control plants grown under greenhouse conditions for two months (Table 2). Samples from mature leaves adjacent to the node at the base of the stem and younger still developing leaves at the top of the stem were analyzed. Plants re-transformed with pMBXS422 (SEQ ID NO: 3) produced up to 7.69% PHB per unit dry weight in samples from mature leaves (Table 2). These are the highest PHB levels reported for monocot biomass crops such as sugarcane, corn and switchgrass. Control plants containing only the PHB genes produced up to 3.53% PHB.

TABLE 2
PHB production in re-transformed switchgrass plants.
Polymer content was measured in mature and developing leaves of vegetative
tillers from plants grown under greenhouse conditions for two months.

| | Number | PHB content [% DW] | | | | | | | |
|---|---|---|---|---|---|---|---|---|---|
| | of plants | Mature leaves | | | | Developing leaves | | | |
| Transgenes | analyzed | Range | Mean | Median | SD | Range | Mean | Median | SD |
| PHB genes | 15 | 0.14-3.53 | 1.44 | 0.96 | 1.06 | 0.18-2.55 | 1.03 | 0.88 | 0.80 |
| PHB genes + | 61 | 0.42-7.69 | 3.48 | 3.19 | 1.52 | 0.00-5.24 | 1.84 | 1.64 | 0.90 |
| pMBXS422 | | | | | | | | | |

### Example 3

Transformation of Switchgrass with the Vectors pMBXS422 and pMBXS424.

Callus cultures were initiated from mature caryopses of cv. “Alamo” following a previously published procedure (Denchev & Conger, (1994), Crop Sci, 34:1623-1627). Cultures were grown at 27° C., in the dark and maintained by monthly subcultures on a fresh medium for callus growth (Somleva et al., (2002), Crop Sci, 42:2080-2087). Their embryogenic potential and plant regeneration ability were evaluated as described previously (U.S. 2009/0271889 A1).

These embryogenic cultures were transformed with Agrobacterium tumefaciens strain AGL1 carrying the binary vector pMBXS424 (SEQ ID NO: 7) in the presence of 100 μM of acetosyringone as previously described (Somleva, (2006), Agrobacterium Protocols, Wang K., ed., pp 65-74, Humana

Press; Somleva et al., (2002), Crop Sci, 42:2080-2087). All infected cultures were selected with 10 mg/L bialaphos for 2 months with transfers to a fresh selection medium every two weeks (Somleva, (2006), Agrobacterium Protocols, Wang K., ed., pp 65-74, Humana Press; Somleva et al., (2002), Crop Sci, 42:2080-2087). Calluses were also transformed with the binary vector pMBXS422 (SEQ ID NO: 3) and selected with 200 mg/L hygromycin for 2 months with monthly transfers to a fresh selection medium.

Bialaphos-resistant calluses from transformations with pMBXS424 (SEQ ID NO: 7) were transferred on to a medium for plant regeneration and selection and the plantlets were treated with the herbicide Basta™ as described previously (Somleva, (2006), Agrobacterium Protocols, Wang K., ed., pp 65-74, Humana Press; Somleva et al., (2002), Crop Sci, 42:2080-2087). Plantlets produced from hygromycin-resistant calluses from transformations with pMBXS422 (SEQ ID NO: 3) were subjected to selection with 200 mg/L of the antibiotic. Non-transformed callus cultures were plated on a regeneration medium and the resultant plantlets were grown under the same in vitro conditions. These wild-type plants served as controls for plant growth rate and biomass accumulation in tissue culture and soil. All regenerants were grown at 27° C. with a 16-h photoperiod (cool white fluorescent bulbs, 80 μmol/m²/s).

The presence of the transgenes in putative transformants was confirmed by PCR as described previously (Somleva et al., (2008), Plant Biotechnol J, 6:663-678; U.S. 2009/0271889 A1) using primers specific for the coding regions of the phaA, phaB, phaC, and FBPase/SBPase genes as well as the marker genes bar and hptII. Transgenic and control plants were grown in a greenhouse at 27° C. with a 16-hour photoperiod with supplemental lighting (sodium halide lamps, 200 μmol/m²/s).

Approximately 23% of the inoculated calluses were bialaphos-resistant at the end of the 2-month-long selection period. Similar results were obtained with the vector harboring only the FBPase/SBPase genes. About 27% of the explants inoculated with pMBXS422 (SEQ ID NO: 3) were hygromycin-resistant and produced at least one transgenic plant.

The PHB content measured in 54 primary transformants in tissue culture was 0-0.42% DW.

### Example 4

### Effects of the Expression of the Synechococcus PCC 7942 FBPase/SBPase Geneon Growth, Development, Biomass Composition, and Photosynthetic Activity of PHB Producing Switchgrass Plants.

### Biomass Accumulation and Plant Development

To determine whether the expression of the FBPase/SBPase gene affected the accumulation of biomass in PHB producing plants, the following experiments were performed. Switchgrass plants obtained by re-transformation of cultures initiated from PHB producing lines with pMBXS422 (SEQ ID NO: 3) (39 plants) were grown under greenhouse conditions for 4 months. Control plants containing only the PHB genes (15 plants) as well as 3 wild-type plants (regenerated from non-transformed immature inflorescence-derived cultures) were also grown under the same conditions. All the plants analyzed in this study are from the “Alamo” genotype 56 (Somleva et al., (2008), Plant Biotechnol J, 6:663-678; U.S. 2009/0271889 A1). At the end of the 4-month period, all vegetative and reproductive tillers at different developmental stages from each plant were counted and cut below the basal node. Leaves and stem tissues were separated, cut into smaller pieces, air-dried at 27° C. for 12-14 days and dry weight measurements were obtained.

The average biomass accumulation in non-transformed (wild-type) plants was 35.5 g dry weight (Table 3). They formed 16-22 tillers and the ratio of vegetative to reproductive tillers was 1:3.

TABLE 3
Effect of the Synechococcus PCC 7942 FBPase/SBPase on growth
and development of PHB producing switchgrass plants.
All plants were grown under greenhouse conditions for 4 months prior to
biomass harvest. The data presented are from measurements of transgenic
plants accumulating more than 1% DW PHB in their mature leaves - 36 out of
39 plants and 7 out of 15 plants for re-transformed and control PHB producers,
respectively.

| | Biomass [g/DW] | | | Number of tillers | | | Plants with >1% PHB | |
|---|---|---|---|---|---|---|---|---|
| Transgenes | Mean | SD | % | Mean | SD | % | Range | % to total |
| WT (control) | 35.5 | 8.3 | 100.0 | 18.0 | 5.7 | 100.0 | N/A | N/A |
| PHB genes | 35.7 | 11.4 | 100.6 | 26.7 | 7.0 | 148.3 | 1.25-3.53 | 46.7 |
| PHB genes + | 33.0 | 17.0 | 93.0 | 27.9 | 10.4 | 155.2 | 1.29-6.41 | 92.3 |
| pMBXS422 | | | | | | | | |

Because of the significant differences in the polymer content in re-transformed and control PHB producers (see Example 2), the data for biomass yield and number of tillers presented in Table 3 are from measurements of transgenic plants accumulating more than 1% DW PHB in their mature leaves (92.3% of the re-transformed and 46.7% of the control PHB producers analyzed) after 2 months growth in soil.

The average biomass production of the control PHB plants was similar to the biomass of the wild type plants, while the yield from the re-transformed plants was reduced with 7% (Table 3). The average ratio of vegetative to reproductive tillers in both groups of PHB producers was 1.2-1.4, which suggested that there were no changes in tiller development compared to wild-type plants. The major difference was the significantly higher tiller formation capacity of the transgenic plants (Table 3).

The total biomass of the micropropagated plant containing only PHB genes with the highest PHB content (3.53% DW) was 23 g dry weight. Re-transformed lines accumulating 3.50-6.41% DW PHB in mature leaves with similar or improved biomass yield up to 48.4 g DW were identified in this study.

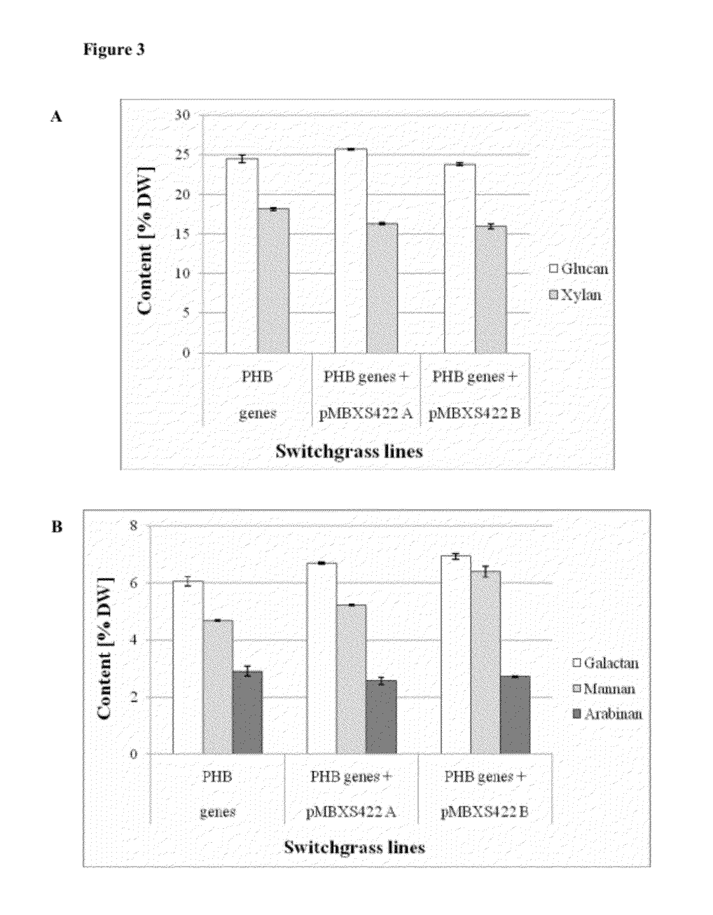

The accumulation of the transgene-encoded proteins in some of these plants was analyzed by Western blots (an example is shown in FIG. 2).

### Biomass Composition

To evaluate the effects of the cyanobacterial FBPase/SBPase gene on biomass composition in PHB producing plants, the contents of starch and structural carbohydrates in leaf tissues were determined.

Starch content: Whole blades of leaves attached to the second node from the base of reproductive tillers (4-5 tillers/plant) with 4 nodes and developing panicles before anthesis were harvested, ground in liquid nitrogen and freeze-dried for 3 days. Resultant leaf powder (40-42 mg/replication) was used for quantitative, enzymatic determination of starch using a Starch Assay Kit (Sigma). PHB content was measured in portions of the powder (20-30 mg dry weight) as described in Example 2.

Both transgenic and wild-type plants used in these experiments were from “Alamo” genotype 56 (Somleva et al., (2008), Plant Biotechnol J, 6:663-678). The PHB producing plants with or without the FBPase/SBPase gene were obtained from immature inflorescence-derived cultures initiated from the same donor plant, thus representing the same transformation event for the PHB genes. The FBPase/SBPase-expressing plants were from independent re-transformation events.

Previously, we have reported the lack of starch granules in leaves of PHB producing primary switchgrass transformants revealed by transmission electron microscopy (Somleva et al., (2008), Plant Biotechnol J, 6:663-678). In this study, significantly reduced starch content compared to wild-type plants was detected in PHB producers obtained from immature inflorescence-derived cultures initiated from a T₀ plant. Total starch amount in the leaves of plants overexpressing the FBPase/SBPase gene was significantly higher than starch content in PHB producing controls (an example is shown in Table 4) suggesting increased photosynthetic capacity. The results also demonstrated the possibility for restoring the primary carbon metabolism in PHB producing switchgrass plants.

TABLE 4
Starch content in leaves from reproductive tillers of soil-grown
PHB producing and wild-type switchgrass plants.

| | Starch content | PHB content |
|---|---|---|
| Transgenes | [% to control] | [% DW] |
| Wild-type control* | 100.0 | 0 |
| PHB genes | 5.3 | 2.4 |
| PHB genes + pMBXS422 A | 68.2 | 2.0 |
| PHB genes + pMBXS422 B | 49.1 | 2.2 |

Both transgenic and wild-type plants are from the same genotype.
All the PHB producing plants represent the same transformation event for the PHB genes.
The PHB genes + pMBXS422 plants A and B areindependent re-transformation events.
*The average starch content in the wild-type leaves was 0.64% DW.

### Structural Carbohydrates Profile in PHB Producing Switchgrass Plants:

Samples (8-10 g dry weight) from total leaf biomass from control and re-transformed PHB producing plants grown under greenhouse conditions for 4 months (see above) were analyzed following standard biomass analytical procedures (http:/www.nrel.gov/biomass). After removal of soluble non-structural materials, samples were subjected to a two-step acid hydrolysis to fractionate the biomass. The monomeric forms of the hydrolyzed polymeric carbohydrates were measured by HPLC.

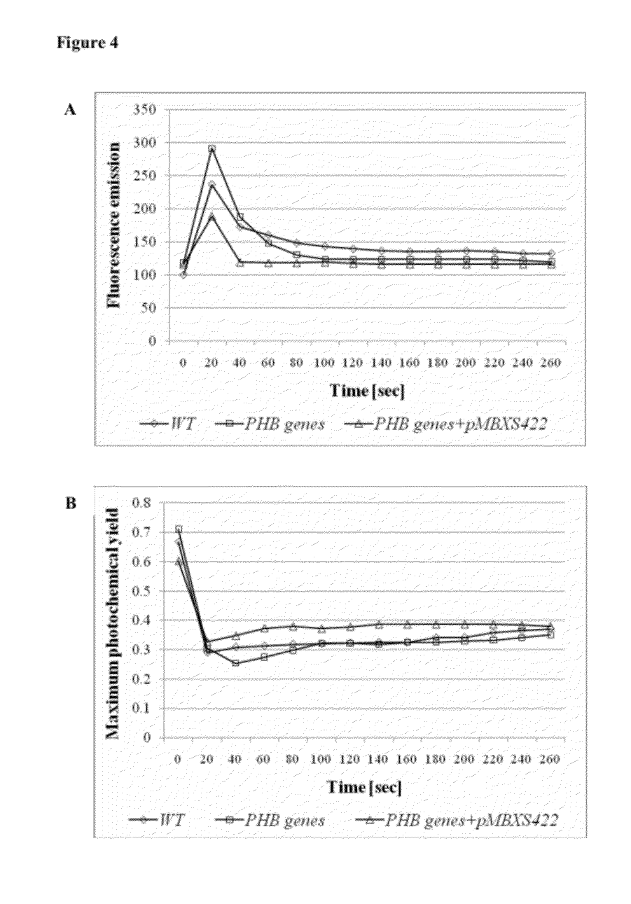

A significant increase in the levels of galactan and mannan combined with significant reduction in xylan content were detected in PHB producing plants re-transformed with the FBPase/SBPase genes compared to control PHB producers (an example is shown in FIG. 3A and B). The former also contained lower levels of arabinan (FIG. 3B). There were no significant differences in the total content of structural carbohydrates (55.8-56.5% dry weight).

The data suggested that the overexpression of the Synechococcus PCC 7942 FBPase/SBPase gene in PHB producing switchgrass plants resulted in significant changes in the levels of some polymeric carbohydrates, which combined with the significantly increased starch content indicated modifications of the biomass composition.

Photosynthetic Parameters of PHB Producing Switchgrass Plants with and without the Expression of the FBPase/SBPase Genes.

For comparative studies of the functioning of the photosystem II (PSII) in light adapted leaves of soil-grown PHB producing plants and PHB producing plants re-transformed with pMBXS422 (SEQ ID NO: 3), the chlorophyll fluorescence, quantum yield of electron transfer, and electron transport rate were measured using a modulated fluorescence system (MONI-PAM). All measurements were performed with the leaf attached to the second node from the base of vegetative tillers with 3-4 visible nodes.

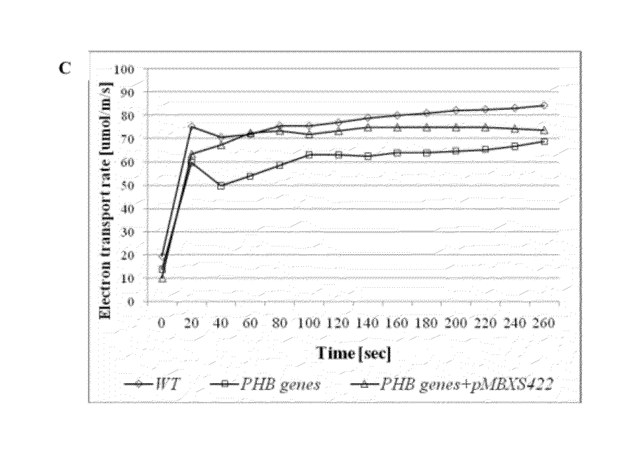

Based on the linear correlation between the quantum yield of PSII and CO₂ fixation in C₄ plants (Leipner et al., (1999), Environ Exp Bot, 42:129-139; Krall & Edwards, (1992), Physiol Plant, 86:180-187), the data for the photosynthetic parameters measured (FIG. 4) suggested that the expression of the FBPase/SBPase gene improved the overall rate of photosynthesis. This suggestion is supported by the significant increase in the PHB production combined with slight reduction of the biomass yield in the re-transformed plants as well as the significantly higher starch content in their leaves.

Other than in the examples herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (i.e., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While this invention has been particularly shown and described with references to preferred embodiments thereof, it will be understood by those skilled in the art that various changes in form and details may be made therein without departing from the scope of the invention encompassed by the appended claims.

| Vector: pMBXS422 | |
|---|---|
| (SEQ ID NO: 3) | |
| 1 | CATGCCAACC ACAGGGTTCC CCTCGGGATC AAAGTACTTT GATCCAACCC |
| 51 | CTCCGCTGCT ATAGTGCAGT CGGCTTCTGA CGTTCAGTGC AGCCGTCTTC |
| 101 | TGAAAACGAC ATGTCGCACA AGTCCTAAGT TACGCGACAG GCTGCCGCCC |
| 151 | TGCCCTTTTC CTGGCGTTTT CTTGTCGCGT GTTTTAGTCG CATAAAGTAG |
| 201 | AATACTTGCG ACTAGAACCG GAGACATTAC GCCATGAACA AGAGCGCCGC |
| 251 | CGCTGGCCTG CTGGGCTATG CCCGCGTCAG CACCGACGAC CAGGACTTGA |
| 301 | CCAACCAACG GGCCGAACTG CACGCGGCCG GCTGCACCAA GCTGTTTTCC |
| 351 | GAGAAGATCA CCGGCACCAG GCGCGACCGC CCGGAGCTGG CCAGGATGCT |
| 401 | TGACCACCTA CGCCCTGGCG ACGTTGTGAC AGTGACCAGG CTAGACCGCC |
| 451 | TGGCCCGCAG CACCCGCGAC CTACTGGACA TTGCCGAGCG CATCCAGGAG |
| 501 | GCCGGCGCGG GCCTGCGTAG CCTGGCAGAG CCGTGGGCCG ACACCACCAC |
| 551 | GCCGGCCGGC CGCATGGTGT TGACCGTGTT CGCCGGCATT GCCGAGTTCG |
| 601 | AGCGTTCCCT AATCATCGAC CGCACCCGGA GCGGGCGCGA GGCCGCCAAG |
| 651 | GCCCGAGGCG TGAAGTTTGG CCCCCGCCCT ACCCTCACCC CGGCACAGAT |
| 701 | CGCGCACGCC CGCGAGCTGA TCGACCAGGA AGGCCGCACC GTGAAAGAGG |
| 751 | CGGCTGCACT GCTTGGCGTG CATCGCTCGA CCCTGTACCG CGCACTTGAG |
| 801 | CGCAGCGAGG AAGTGACGCC CACCGAGGCC AGGCGGCGCG GTGCCTTCCG |
| 851 | TGAGGACGCA TTGACCGAGG CCGACGCCCT GGCGGCCGCC GAGAATGAAC |
| 901 | GCCAAGAGGA ACAAGCATGA AACCGCACCA GGACGGCCAG GACGAACCGT |
| 951 | TTTTCATTAC CGAAGAGATC GAGGCGGAGA TGATCGCGGC CGGGTACGTG |
| 1001 | TTCGAGCCGC CCGCGCACGT CTCAACCGTG CGGCTGCATG AAATCCTGGC |
| 1051 | CGGTTTGTCT GATGCCAAGC TGGCGGCCTG GCCGGCCAGC TTGGCCGCTG |
| 1101 | AAGAAACCGA GCGCCGCCGT CTAAAAAGGT GATGTGTATT TGAGTAAAAC |
| 1151 | AGCTTGCGTC ATGCGGTCGC TGCGTATATG ATGCGATGAG TAAATAAACA |
| 1201 | AATACGCAAG GGGAACGCAT GAAGGTTATC GCTGTACTTA ACCAGAAAGG |
| 1251 | CGGGTCAGGC AAGACGACCA TCGCAACCCA TCTAGCCCGC GCCCTGCAAC |
| 1301 | TCGCCGGGGC CGATGTTCTG TTAGTCGATT CCGATCCCCA GGGCAGTGCC |
| 1351 | CGCGATTGGG CGGCCGTGCG GGAAGATCAA CCGCTAACCG TTGTCGGCAT |
| 1401 | CGACCGCCCG ACGATTGACC GCGACGTGAA GGCCATCGGC CGGCGCGACT |
| 1451 | TCGTAGTGAT CGACGGAGCG CCCCAGGCGG CGGACTTGGC TGTGTCCGCG |
| 1501 | ATCAAGGCAG CCGACTTCGT GCTGATTCCG GTGCAGCCAA GCCCTTACGA |
| 1551 | CATATGGGCC ACCGCCGACC TGGTGGAGCT GGTTAAGCAG CGCATTGAGG |
| 1601 | TCACGGATGG AAGGCTACAA GCGGCCTTTG TCGTGTCGCG GGCGATCAAA |
| 1651 | GGCACGCGCA TCGGCGGTGA GGTTGCCGAG GCGCTGGCCG GGTACGAGCT |
| 1701 | GCCCATTCTT GAGTCCCGTA TCACGCAGCG CGTGAGCTAC CCAGGCACTG |
| 1751 | CCGCCGCCGG CACAACCGTT CTTGAATCAG AACCCGAGGG CGACGCTGCC |
| 1801 | CGCGAGGTCC AGGCGCTGGC CGCTGAAATT AAATCAAAAC TCATTTGAGT |
| 1851 | TAATGAGGTA AAGAGAAAAT GAGCAAAAGC ACAAACACGC TAAGTGCCGG |
| 1901 | CCGTCCGAGC GCACGCAGCA GCAAGGCTGC AACGTTGGCC AGCCTGGCAG |
| 1951 | ACACGCCAGC CATGAAGCGG GTCAACTTTC AGTTGCCGGC GGAGGATCAC |
| 2001 | ACCAAGCTGA AGATGTACGC GGTACGCCAA GGCAAGACCA TTACCGAGCT |
| 2051 | GCTATCTGAA TACATCGCGC AGCTACCAGA GTAAATGAGC AAATGAATAA |
| 2101 | ATGAGTAGAT GAATTTTAGC GGCTAAAGGA GGCGGCATGG AAAATCAAGA |
| 2151 | ACAACCAGGC ACCGACGCCG TGGAATGCCC CATGTGTGGA GGAACGGGCG |
| 2201 | GTTGGCCAGG CGTAAGCGGC TGGGTTGTCT GCCGGCCCTG CAATGGCACT |
| 2251 | GGAACCCCCA AGCCCGAGGA ATCGGCGTGA CGGTCGCAAA CCATCCGGCC |
| 2301 | CGGTACAAAT CGGCGCGGCG CTGGGTGATG ACCTGGTGGA GAAGTTGAAG |
| 2351 | GCCGCGCAGG CCGCCCAGCG GCAACGCATC GAGGCAGAAG CACGCCCCGG |
| 2401 | TGAATCGTGG CAAGCGGCCG CTGATCGAAT CCGCAAAGAA TCCCGGCAAC |
| 2451 | CGCCGGCAGC CGGTGCGCCG TCGATTAGGA AGCCGCCCAA GGGCGACGAG |
| 2501 | CAACCAGATT TTTTCGTTCC GATGCTCTAT GACGTGGGCA CCCGCGATAG |
| 2551 | TCGCAGCATC ATGGACGTGG CCGTTTTCCG TCTGTCGAAG CGTGACCGAC |
| 2601 | GAGCTGGCGA GGTGATCCGC TACGAGCTTC CAGACGGGCA CGTAGAGGTT |
| 2651 | TCCGCAGGGC CGGCCGGCAT GGCCAGTGTG TGGGATTACG ACCTGGTACT |
| 2701 | GATGGCGGTT TCCCATCTAA CCGAATCCAT GAACCGATAC CGGGAAGGGA |
| 2751 | AGGGAGACAA GCCCGGCCGC GTGTTCCGTC CACACGTTGC GGACGTACTC |
| 2801 | AAGTTCTGCC GGCGAGCCGA TGGCGGAAAG CAGAAAGACG ACCTGGTAGA |
| 2851 | AACCTGCATT CGGTTAAACA CCACGCACGT TGCCATGCAG CGTACGAAGA |
| 2901 | AGGCCAAGAA CGGCCGCCTG GTGACGGTAT CCGAGGGTGA AGCCTTGATT |
| 2951 | AGCCGCTACA AGATCGTAAA GAGCGAAACC GGGCGGCCGG AGTACATCGA |
| 3001 | GATCGAGCTA GCTGATTGGA TGTACCGCGA GATCACAGAA GGCAAGAACC |
| 3051 | CGGACGTGCT GACGGTTCAC CCCGATTACT TTTTGATCGA TCCCGGCATC |
| 3101 | GGCCGTTTTC TCTACCGCCT GGCACGCCGC GCCGCAGGCA AGGCAGAAGC |
| 3151 | CAGATGGTTG TTCAAGACGA TCTACGAACG CAGTGGCAGC GCCGGAGAGT |
| 3201 | TCAAGAAGTT CTGTTTCACC GTGCGCAAGC TGATCGGGTC AAATGACCTG |
| 3251 | CCGGAGTACG ATTTGAAGGA GGAGGCGGGG CAGGCTGGCC CGATCCTAGT |
| 3301 | CATGCGCTAC CGCAACCTGA TCGAGGGCGA AGCATCCGCC GGTTCCTAAT |
| 3351 | GTACGGAGCA GATGCTAGGG CAAATTGCCC TAGCAGGGGA AAAAGGTCGA |
| 3401 | AAAGGTCTCT TTCCTGTGGA TAGCACGTAC ATTGGGAACC CAAAGCCGTA |
| 3451 | CATTGGGAAC CGGAACCCGT ACATTGGGAA CCCAAAGCCG TACATTGGGA |
| 3501 | ACCGGTCACA CATGTAAGTG ACTGATATAA AAGAGAAAAA AGGCGATTTT |
| 3551 | TCCGCCTAAA ACTCTTTAAA ACTTATTAAA ACTCTTAAAA CCCGCCTGGC |
| 3601 | CTGTGCATAA CTGTCTGGCC AGCGCACAGC CGAAGAGCTG CAAAAAGCGC |
| 3651 | CTACCCTTCG GTCGCTGCGC TCCCTACGCC CCGCCGCTTC GCGTCGGCCT |
| 3701 | ATCGCGGCCG CTGGCCGCTC AAAAATGGCT GGCCTACGGC CAGGCAATCT |
| 3751 | ACCAGGGCGC GGACAAGCCG CGCCGTCGCC ACTCGACCGC CGGCGCCCAC |
| 3801 | ATCAAGGCAC CCTGCCTCGC GCGTTTCGGT GATGACGGTG AAAACCTCTG |
| 3851 | ACACATGCAG CTCCCGGAGA CGGTCACAGC TTGTCTGTAA GCGGATGCCG |
| 3901 | GGAGCAGACA AGCCCGTCAG GGCGCGTCAG CGGGTGTTGG CGGGTGTCGG |
| 3951 | GGCGCAGCCA TGACCCAGTC ACGTAGCGAT AGCGGAGTGT ATACTGGCTT |
| 4001 | AACTATGCGG CATCAGAGCA GATTGTACTG AGAGTGCACC ATATGCGGTG |
| 4051 | TGAAATACCG CACAGATGCG TAAGGAGAAA ATACCGCATC AGGCGCTCTT |
| 4101 | CCGCTTCCTC GCTCACTGAC TCGCTGCGCT CGGTCGTTCG GCTGCGGCGA |
| 4151 | GCGGTATCAG CTCACTCAAA GGCGGTAATA CGGTTATCCA CAGAATCAGG |
| 4201 | GGATAACGCA GGAAAGAACA TGTGAGCAAA AGGCCAGCAA AAGGCCAGGA |
| 4251 | ACCGTAAAAA GGCCGCGTTG CTGGCGTTTT TCCATAGGCT CCGCCCCCCT |
| 4301 | GACGAGCATC ACAAAAATCG ACGCTCAAGT CAGAGGTGGC GAAACCCGAC |
| 4351 | AGGACTATAA AGATACCAGG CGTTTCCCCC TGGAAGCTCC CTCGTGCGCT |
| 4401 | CTCCTGTTCC GACCCTGCCG CTTACCGGAT ACCTGTCCGC CTTTCTCCCT |
| 4451 | TCGGGAAGCG TGGCGCTTTC TCATAGCTCA CGCTGTAGGT ATCTCAGTTC |
| 4501 | GGTGTAGGTC GTTCGCTCCA AGCTGGGCTG TGTGCACGAA CCCCCCGTTC |
| 4551 | AGCCCGACCG CTGCGCCTTA TCCGGTAACT ATCGTCTTGA GTCCAACCCG |
| 4601 | GTAAGACACG ACTTATCGCC ACTGGCAGCA GCCACTGGTA ACAGGATTAG |
| 4651 | CAGAGCGAGG TATGTAGGCG GTGCTACAGA GTTCTTGAAG TGGTGGCCTA |
| 4701 | ACTACGGCTA CACTAGAAGG ACAGTATTTG GTATCTGCGC TCTGCTGAAG |
| 4751 | CCAGTTACCT TCGGAAAAAG AGTTGGTAGC TCTTGATCCG GCAAACAAAC |
| 4801 | CACCGCTGGT AGCGGTGGTT TTTTTGTTTG CAAGCAGCAG ATTACGCGCA |
| 4851 | GAAAAAAAGG ATCTCAAGAA GATCCTTTGA TCTTTTCTAC GGGGTCTGAC |
| 4901 | GCTCAGTGGA ACGAAAACTC ACGTTAAGGG ATTTTGGTCA TGCATTCTAG |
| 4951 | GTACTAAAAC AATTCATCCA GTAAAATATA ATATTTTATT TTCTCCCAAT |
| 5001 | CAGGCTTGAT CCCCAGTAAG TCAAAAAATA GCTCGACATA CTGTTCTTCC |
| 5051 | CCGATATCCT CCCTGATCGA CCGGACGCAG AAGGCAATGT CATACCACTT |
| 5101 | GTCCGCCCTG CCGCTTCTCC CAAGATCAAT AAAGCCACTT ACTTTGCCAT |
| 5151 | CTTTCACAAA GATGTTGCTG TCTCCCAGGT CGCCGTGGGA AAAGACAAGT |
| 5201 | TCCTCTTCGG GCTTTTCCGT CTTTAAAAAA TCATACAGCT CGCGCGGATC |
| 5251 | TTTAAATGGA GTGTCTTCTT CCCAGTTTTC GCAATCCACA TCGGCCAGAT |
| 5301 | CGTTATTCAG TAAGTAATCC AATTCGGCTA AGCGGCTGTC TAAGCTATTC |
| 5351 | GTATAGGGAC AATCCGATAT GTCGATGGAG TGAAAGAGCC TGATGCACTC |
| 5401 | CGCATACAGC TCGATAATCT TTTCAGGGCT TTGTTCATCT TCATACTCTT |
| 5451 | CCGAGCAAAG GACGCCATCG GCCTCACTCA TGAGCAGATT GCTCCAGCCA |
| 5501 | TCATGCCGTT CAAAGTGCAG GACCTTTGGA ACAGGCAGCT TTCCTTCCAG |
| 5551 | CCATAGCATC ATGTCCTTTT CCCGTTCCAC ATCATAGGTG GTCCCTTTAT |
| 5601 | ACCGGCTGTC CGTCATTTTT AAATATAGGT TTTCATTTTC TCCCACCAGC |
| 5651 | TTATATACCT TAGCAGGAGA CATTCCTTCC GTATCTTTTA CGCAGCGGTA |
| 5701 | TTTTTCGATC AGTTTTTTCA ATTCCGGTGA TATTCTCATT TTAGCCATTT |
| 5751 | ATTATTTCCT TCCTCTTTTC TACAGTATTT AAAGATACCC CAAGAAGCTA |
| 5801 | ATTATAACAA GACGAACTCC AATTCACTGT TCCTTGCATT CTAAAACCTT |
| 5851 | AAATACCAGA AAACAGCTTT TTCAAAGTTG TTTTCAAAGT TGGCGTATAA |
| 5901 | CATAGTATCG ACGGAGCCGA TTTTGAAACC GCGGTGATCA CAGGCAGCAA |
| 5951 | CGCTCTGTCA TCGTTACAAT CAACATGCTA CCCTCCGCGA GATCATCCGT |
| 6001 | GTTTCAAACC CGGCAGCTTA GTTGCCGTTC TTCCGAATAG CATCGGTAAC |
| 6051 | ATGAGCAAAG TCTGCCGCCT TACAACGGCT CTCCCGCTGA CGCCGTCCCG |
| 6101 | GACTGATGGG CTGCCTGTAT CGAGTGGTGA TTTTGTGCCG AGCTGCCGGT |
| 6151 | CGGGGAGCTG TTGGCTGGCT GGTGGCAGGA TATATTGTGG TGTAAACAAA |
| 6201 | TTGACGCTTA GACAACTTAA TAACACATTG CGGACGTTTT TAATGTACTG |
| 6251 | AATTAACGCC GAATTAATTC GGGGGATCTG GATTTTAGTA CTGGATTTTG |
| 6301 | GTTTTAGGAA TTAGAAATTT TATTGATAGA AGTATTTTAC AAATACAAAT |
| 6351 | ACATACTAAG GGTTTCTTAT ATGCTCAACA CATGAGCGAA ACCCTATAGG |
| 6401 | AACCCTAATT CCCTTATCTG GGAACTACTC ACACATTATT ATGGAGAAAC |
| 6451 | TCGAGGGATC CCGGTCGGCA TCTACTCTAT TCCTTTGCCC TCGGACGAGT |
| 6501 | GCTGGGGCGT CGGTTTCCAC TATCGGCGAG TACTTCTACA CAGCCATCGG |
| 6551 | TCCAGACGGC CGCGCTTCTG CGGGCGATTT GTGTACGCCC GACAGTCCCG |
| 6601 | GCTCCGGATC GGACGATTGC GTCGCATCGA CCCTGCGCCC AAGCTGCATC |
| 6651 | ATCGAAATTG CCGTCAACCA AGCTCTGATA GAGTTGGTCA AGACCAATGC |
| 6701 | GGAGCATATA CGCCCGGAGC CGCGGCGATC CTGCAAGCTC CGGATGCCTC |
| 6751 | CGCTCGAAGT AGCGCGTCTG CTGCTCCATA CAAGCCAACC ACGGCCTCCA |
| 6801 | GAAGAAGATG TTGGCGACCT CGTATTGGGA ATCCCCGAAC ATCGCCTCGC |
| 6851 | TCCAGTCAAT GACCGCTGTT ATGCGGCCAT TGTCCGTCAG GACATTGTTG |
| 6901 | GAGCCGAAAT CCGCGTGCAC GAGGTGCCGG ACTTCGGGGC AGTCCTCGGC |
| 6951 | CCAAAGCATC AGCTCATCGA GAGCCTGCGC GACGGACGCA CTGACGGTGT |
| 7001 | CGTCCATCAC AGTTTGCCAG TGATACACAT GGGGATCAGC AATCGCGCAT |
| 7051 | ATGAAATCAC GCCATGTAGT GTATTGACCG ATTCCTTGCG GTCCGAATGG |
| 7101 | GCCGAACCCG CTCGTCTGGC TAAGATCGGC CGCAGCGATC GCATCCATGG |
| 7151 | CCTCCGCGAC CGGCTGCAGT TATCATCATC ATCATAGACA CACGAAATAA |
| 7201 | AGTAATCAGA TTATCAGTTA AAGCTATGTA ATATTTACAC CATAACCAAT |
| 7251 | CAATTAAAAA ATAGATCAGT TTAAAGAAAG ATCAAAGCTC AAAAAAATAA |
| 7301 | AAAGAGAAAA GGGTCCTAAC CAAGAAAATG AAGGAGAAAA ACTAGAAATT |
| 7351 | TACCTGCAGA ACAGCGGGCA GTTCGGTTTC AGGCAGGTCT TGCAACGTGA |
| 7401 | CACCCTGTGC ACGGCGGGAG ATGCAATAGG TCAGGCTCTC GCTGAATTCC |
| 7451 | CCAATGTCAA GCACTTCCGG AATCGGGAGC GCGGCCGATG CAAAGTGCCG |
| 7501 | ATAAACATAA CGATCTTTGT AGAAACCATC GGCGCAGCTA TTTACCCGCA |
| 7551 | GGACATATCC ACGCCCTCCT ACATCGAAGC TGAAAGCACG AGATTCTTCG |
| 7601 | CCCTCCGAGA GCTGCATCAG GTCGGAGACG CTGTCGAACT TTTCGATCAG |
| 7651 | AAACTTCTCG ACAGACGTCG CGGTGAGTTC AGGCTTTTTC ATGGTAGAGG |
| 7701 | AGCTCGCCGC TTGGTATCTG CATTACAATG AAATGAGCAA AGACTATGTG |
| 7751 | AGTAACACTG GTCAACACTA GGGAGAAGGC ATCGAGCAAG ATACGTATGT |
| 7801 | AAAGAGAAGC AATATAGTGT CAGTTGGTAG ATACTAGATA CCATCAGGAG |
| 7851 | GTAAGGAGAG CAACAAAAAG GAAACTCTTT ATTTTTAAAT TTTGTTACAA |
| 7901 | CAAACAAGCA GATCAATGCA TCAAAATACT GTCAGTACTT ATTTCTTCAG |
| 7951 | ACAACAATAT TTAAAACAAG TGCATCTGAT CTTGACTTAT GGTCACAATA |
| 8001 | AAGGAGCAGA GATAAACATC AAAATTTCGT CATTTATATT TATTCCTTCA |
| 8051 | GGCGTTAACA ATTTAACAGC ACACAAACAA AAACAGAATA GGAATATCTA |
| 8101 | ATTTTGGCAA ATAATAAGCT CTGCAGACGA ACAAATTATT ATAGTATCGC |
| 8151 | CTATAATATG AATCCCTATA CTATTGACCC ATGTAGTATG AAGCCTGTGC |
| 8201 | CTAAATTAAC AGCAAACTTC TGAATCCAAG TGCCCTATAA CACCAACATG |
| 8251 | TGCTTAAATA AATACCGCTA AGCACCAAAT TACACATTTC TCGTATTGCT |
| 8301 | GTGTAGGTTC TATCTTCGTT TCGTACTACC ATGTCCCTAT ATTTTGCTGC |
| 8351 | TACAAAGGAC GGCAAGTAAT CAGCACAGGC AGAACACGAT TTCAGAGTGT |
| 8401 | AATTCTAGAT CCAGCTAAAC CACTCTCAGC AATCACCACA CAAGAGAGCA |
| 8451 | TTCAGAGAAA CGTGGCAGTA ACAAAGGCAG AGGGCGGAGT GAGCGCGTAC |
| 8501 | CGAAGACGGT AGATCTCTCG AGAGAGATAG ATTTGTAGAG AGAGACTGGT |
| 8551 | GATTTCAGCG TGTCCTCTCC AAATGAAATG AACTTCCTTA TATAGAGGAA |
| 8601 | GGTCTTGCGA AGGATAGTGG GATTGTGCGT CATCCCTTAC GTCAGTGGAG |
| 8651 | ATATCACATC AATCCACTTG CTTTGAAGAC GTGGTTGGAA CGTCTTCTTT |
| 8701 | TTCCACGATG CTCCTCGTGG GTGGGGGTCC ATCTTTGGGA CCACTGTCGG |
| 8751 | CAGAGGCATC TTGAACGATA GCCTTTCCTT TATCGCAATG ATGGCATTTG |
| 8801 | TAGGTGCCAC CTTCCTTTTC TACTGTCCTT TTGATGAAGT GACAGATAGC |
| 8851 | TGGGCAATGG AATCCGAGGA GGTTTCCCGA TATTACCCTT TGTTGAAAAG |
| 8901 | TCTCAATAGC CCTTTGGTCT TCTGAGACTG TATCTTTGAT ATTCTTGGAG |
| 8951 | TAGACGAGAG TGTCGTGCTC CACCATGTTA TCACATCAAT CCACTTGCTT |
| 9001 | TGAAGACGTG GTTGGAACGT CTTCTTTTTC CACGATGCTC CTCGTGGGTG |
| 9051 | GGGGTCCATC TTTGGGACCA CTGTCGGCAG AGGCATCTTG AACGATAGCC |
| 9101 | TTTCCTTTAT CGCAATGATG GCATTTGTAG GTGCCACCTT CCTTTTCTAC |
| 9151 | TGTCCTTTTG ATGAAGTGAC AGATAGCTGG GCAATGGAAT CCGAGGAGGT |
| 9201 | TTCCCGATAT TACCCTTTGT TGAAAAGTCT CAATAGCCCT TTGGTCTTCT |
| 9251 | GAGACTGTAT CTTTGATATT CTTGGAGTAG ACGAGAGTGT CGTGCTCCAC |
| 9301 | CATGTTGGCA AGCTGCTCTA GCCAATACGC AAACCGCCTC TCCCCGCGCG |
| 9351 | TTGGCCGATT CATTAATGCA GCTGGCACGA CAGGTTTCCC GACTGGAAAG |
| 9401 | CGGGCAGTGA GCGCAACGCA ATTAATGTGA GTTAGCTCAC TCATTAGGCA |
| 9451 | CCCCAGGCTT TACACTTTAT GCTTCCGGCT CGTATGTTGT GTGGAATTGT |
| 9501 | GAGCGGATAA CAATTTCACA CAGGAAACAG CTATGACCAT GATTACGAAT |
| 9551 | TCGAGCTCGG TACCCCACGG AAGATCCAGG TCTCGAGACT AGGAGACGGA |
| 9601 | TGGGAGGCGC AACGCGCGAT GGGGAGGGGG GCGGCGCTGA CCTTTCTGGC |
| 9651 | GAGGTCGAGG TAGCGATCGA GCAGCTGCAG CGCGGACACG ATGAGGAAGA |
| 9701 | CGAAGATAGC CGCCATGGAC ATGTTCGCCA GCGGCGGCGG AGCGAGGCTG |
| 9751 | AGCCGGTCTC TCCGGCCTCC GGTCGGCGTT AAGTTGGGGA TCGTAACGTG |
| 9801 | ACGTGTCTCG TCTCCACGGA TCGACACAAC CGGCCTACTC GGGTGCACGA |
| 9851 | CGCCGCGATA AGGGCGAGAT GTCCGTGCAC GCAGCCCGTT TGGAGTCCTC |
| 9901 | GTTGCCCACG AACCGACCCC TTACAGAACA AGGCCTAGCC CAAAACTATT |
| 9951 | CTGAGTTGAG CTTTTGAGCC TAGCCCACCT AAGCCGAGCG TCATGAACTG |
| 10001 | ATGAACCCAC TACCACTAGT CAAGGCAAAC CACAACCACA AATGGATCAA |
| 10051 | TTGATCTAGA ACAATCCGAA GGAGGGGAGG CCACGTCACA CTCACACCAA |
| 10101 | CCGAAATATC TGCCAGAATC AGATCAACCG GCCAATAGGA CGCCAGCGAG |
| 10151 | CCCAACACCT GGCGACGCCG CAAAATTCAC CGCGAGGGGC ACCGGGCACG |
| 10201 | GCAAAAACAA AAGCCCGGCG CGGTGAGAAT ATCTGGCGAC TGGCGGAGAC |
| 10251 | CTGGTGGCCA GCGCGCGGCC ACATCAGCCA CCCCATCCGC CCACCTCACC |
| 10301 | TCCGGCGAGC CAATGGCAAC TCGTCTTAAG ATTCCACGAG ATAAGGACCC |
| 10351 | GATCGCCGGC GACGCTATTT AGCCAGGTGC GCCCCCCACG GTACACTCCA |
| 10401 | CCAGCGGCAT CTATAGCAAC CGGTCCAGCA CTTTCACGCT CAGCTTCAGC |
| 10451 | AAGATCTACC GTCTTCGGTA CGCGCTCACT CCGCCCTCTG CCTTTGTTAC |
| 10501 | TGCCACGTTT CTCTGAATGC TCTCTTGTGT GGTGATTGCT GAGAGTGGTT |
| 10551 | TAGCTGGATC TAGAATTACA CTCTGAAATC GTGTTCTGCC TGTGCTGATT |
| 10601 | ACTTGCCGTC CTTTGTAGCA GCAAAATATA GGGACATGGT AGTACGAAAC |
| 10651 | GAAGATAGAA CCTACACAGC AATACGAGAA ATGTGTAATT TGGTGCTTAG |
| 10701 | CGGTATTTAT TTAAGCACAT GTTGGTGTTA TAGGGCACTT GGATTCAGAA |
| 10751 | GTTTGCTGTT AATTTAGGCA CAGGCTTCAT ACTACATGGG TCAATAGTAT |
| 10801 | AGGGATTCAT ATTATAGGCG ATACTATAAT AATTTGTTCG TCTGCAGAGC |
| 10851 | TTATTATTTG CCAAAATTAG ATATTCCTAT TCTGTTTTTG TTTGTGTGCT |
| 10901 | GTTAAATTGT TAACGCCTGA AGGAATAAAT ATAAATGACG AAATTTTGAT |
| 10951 | GTTTATCTCT GCTCCTTTAT TGTGACCATA AGTCAAGATC AGATGCACTT |
| 11001 | GTTTTAAATA TTGTTGTCTG AAGAAATAAG TACTGACAGT ATTTTGATGC |
| 11051 | ATTGATCTGC TTGTTTGTTG TAACAAAATT TAAAAATAAA GAGTTTCCTT |
| 11101 | TTTGTTGCTC TCCTTACCTC CTGATGGTAT CTAGTATCTA CCAACTGATA |
| 11151 | CTATATTGCT TCTCTTTACA NNNNNNTCTT GCTCGATGCC TTCTCCTAGT |
| 11201 | GTTGACCAGT GTTACTCACA TAGTCTTTGC TCATTTCATT GTAATGCAGA |
| 11251 | TACCAAGCGG TTAATTAAAA ATGGCTTCTA TGATATCCTC TTCCGCTGTG |
| 11301 | ACAACAGTCA GCCGTGCCTC TAGGGGGCAA TCCGCCGCAG TGGCTCCATT |
| 11351 | CGGCGGCCTC AAATCCATGA CTGGATTCCC AGTGAAGAAG GTCAACACTG |
| 11401 | ACATTACTTC CATTACAAGC AATGGTGGAA GAGTAAAGTG CATGCAGGTG |
| 11451 | TGGCCTCCAA TTGGAAAGAA GAAGTTTGAG ACTCTTTCCT ATTTGCCACC |
| 11501 | ATTGACGAGA GATTCTAGAG TGGAGAAGAC GATCGGTCTC GAGATTATTG |
| 11551 | AAGTTGTCGA GCAGGCAGCG ATCGCCTCGG CCCGCCTGAT GGGCAAAGGC |
| 11601 | GAAAAGAATG AAGCCGATCG CGTCGCAGTA GAAGCGATGC GGGTGCGGAT |
| 11651 | GAACCAAGTG GAAATGCTGG GCCGCATCGT CATCGGTGAA GGCGAGCGCG |
| 11701 | ACGAAGCACC GATGCTCTAT ATCGGTGAAG AAGTGGGCAT CTACCGCGAT |
| 11751 | GCAGACAAGC GGGCTGGCGT ACCGGCTGGC AAGCTGGTGG AAATCGACAT |
| 11801 | CGCCGTTGAC CCCTGCGAAG GCACCAACCT CTGCGCCTAC GGTCAGCCCG |
| 11851 | GCTCGATGGC AGTTTTGGCC ATCTCCGAGA AAGGCGGCCT GTTTGCAGCT |
| 11901 | CCCGACTTCT ACATGAAGAA ACTGGCTGCA CCCCCAGCTG CCAAAGGCAA |
| 11951 | AGTAGACATC AATAAGTCCG CGACCGAAAA CCTGAAAATT CTCTCGGAAT |
| 12001 | GTCTCGATCG CGCCATCGAT GAATTGGTGG TCGTGGTCAT GGATCGTCCC |
| 12051 | CGCCACAAAG AGCTAATCCA AGAGATCCGC CAAGCGGGTG CCCGCGTCCG |
| 12101 | TCTGATCAGC GATGGTGACG TTTCGGCCGC GATCTCCTGC GGTTTTGCTG |
| 12151 | GCACCAACAC CCACGCCCTG ATGGGCATCG GTGCAGCTCC CGAGGGTGTG |
| 12201 | ATTTCGGCAG CAGCAATGCG TTGCCTCGGC GGTCACTTCC AAGGCCAGCT |
| 12251 | GATCTACGAC CCAGAAGTGG TCAAAACCGG CCTGATCGGT GAAAGCCGTG |
| 12301 | AGAGCAACAT CGCTCGCCTG CAAGAAATGG GCATCACCGA TCCCGATCGC |
| 12351 | GTCTACGACG CCAACGAACT GGCTTCGGGT CAAGAAGTGC TGTTTGCGGC |
| 12401 | TTGCGGTATC ACCCCGGGCT TGCTGATGGA AGGCGTGCGC TTCTTCAAAG |
| 12451 | GCGGCGCTCG CACCCAGAGC TTGGTGATCT CCAGCCAGTC ACGGACGGCT |
| 12501 | CGCTTCGTTG ACACCGTTCA CATGTTCGAC GATGTCAAAA CGGTTAGCCT |
| 12551 | CCGTTAACTG CAGGGCGCGC CATCGTTCAA ACATTTGGCA ATAAAGTTTC |
| 12601 | TTAAGATTGA ATCCTGTTGC CGGTCTTGCG ATGATTATCA TATAATTTCT |
| 12651 | GTTGAATTAC GTTAAGCATG TAATAATTAA CATGTAATGC ATGACGTTAT |
| 12701 | TTATGAGATG GGTTTTTATG ATTAGAGTCC CGCAATTATA CATTTAATAC |
| 12751 | GCGATAGAAA ACAAAATATA GCGCGCAAAC TAGGATAAAT TATCGCGCGC |
| 12801 | GGTGTCATCT ATGTTACTAG ATCCGATGAT AAGCTGTCAA ACATGAAAGC |
| 12851 | TTGGCACTGG CCGTCGTTTT ACAACGTCGT GACTGGGAAA ACCCTGGCGT |
| 12901 | TACCCAACTT AATCGCCTTG CAGCACATCC CCCTTTCGCC AGCTGGCGTA |
| 12951 | ATAGCGAAGA GGCCCGCACC GATCGCCCTT CCCAACAGTT GCGCAGCCTG |
| 13001 | AATGGCGAAT GCTAGAGCAG CTTGAGCTTG GATCAGATTG TCGTTTCCCG |
| 13051 | CCTTCAGTTT AAACTATCAG TGTTTGACAG GATATATTGG CGGGTAAACC |
| 13101 | TAAGAGAAAA GAGCGTTTAT TAGAATAACG GATATTTAAA AGGGCGTGAA |
| 13151 | AAGGTTTATC CGTTCGTCCA TTTGTATGTG |
| Vector: pMBXS424 | |
| (SEQ ID NO: 7) | |
| 1 | CATGCCAACC ACAGGGTTCC CCTCGGGATC AAAGTACTTT GATCCAACCC |
| 51 | CTCCGCTGCT ATAGTGCAGT CGGCTTCTGA CGTTCAGTGC AGCCGTCTTC |
| 101 | TGAAAACGAC ATGTCGCACA AGTCCTAAGT TACGCGACAG GCTGCCGCCC |
| 151 | TGCCCTTTTC CTGGCGTTTT CTTGTCGCGT GTTTTAGTCG CATAAAGTAG |
| 201 | AATACTTGCG ACTAGAACCG GAGACATTAC GCCATGAACA AGAGCGCCGC |
| 251 | CGCTGGCCTG CTGGGCTATG CCCGCGTCAG CACCGACGAC CAGGACTTGA |
| 301 | CCAACCAACG GGCCGAACTG CACGCGGCCG GCTGCACCAA GCTGTTTTCC |
| 351 | GAGAAGATCA CCGGCACCAG GCGCGACCGC CCGGAGCTGG CCAGGATGCT |
| 401 | TGACCACCTA CGCCCTGGCG ACGTTGTGAC AGTGACCAGG CTAGACCGCC |
| 451 | TGGCCCGCAG CACCCGCGAC CTACTGGACA TTGCCGAGCG CATCCAGGAG |
| 501 | GCCGGCGCGG GCCTGCGTAG CCTGGCAGAG CCGTGGGCCG ACACCACCAC |
| 551 | GCCGGCCGGC CGCATGGTGT TGACCGTGTT CGCCGGCATT GCCGAGTTCG |
| 601 | AGCGTTCCCT AATCATCGAC CGCACCCGGA GCGGGCGCGA GGCCGCCAAG |
| 651 | GCCCGAGGCG TGAAGTTTGG CCCCCGCCCT ACCCTCACCC CGGCACAGAT |
| 701 | CGCGCACGCC CGCGAGCTGA TCGACCAGGA AGGCCGCACC GTGAAAGAGG |
| 751 | CGGCTGCACT GCTTGGCGTG CATCGCTCGA CCCTGTACCG CGCACTTGAG |
| 801 | CGCAGCGAGG AAGTGACGCC CACCGAGGCC AGGCGGCGCG GTGCCTTCCG |
| 851 | TGAGGACGCA TTGACCGAGG CCGACGCCCT GGCGGCCGCC GAGAATGAAC |
| 901 | GCCAAGAGGA ACAAGCATGA AACCGCACCA GGACGGCCAG GACGAACCGT |
| 951 | TTTTCATTAC CGAAGAGATC GAGGCGGAGA TGATCGCGGC CGGGTACGTG |
| 1001 | TTCGAGCCGC CCGCGCACGT CTCAACCGTG CGGCTGCATG AAATCCTGGC |
| 1051 | CGGTTTGTCT GATGCCAAGC TGGCGGCCTG GCCGGCCAGC TTGGCCGCTG |
| 1101 | AAGAAACCGA GCGCCGCCGT CTAAAAAGGT GATGTGTATT TGAGTAAAAC |
| 1151 | AGCTTGCGTC ATGCGGTCGC TGCGTATATG ATGCGATGAG TAAATAAACA |
| 1201 | AATACGCAAG GGGAACGCAT GAAGGTTATC GCTGTACTTA ACCAGAAAGG |
| 1251 | CGGGTCAGGC AAGACGACCA TCGCAACCCA TCTAGCCCGC GCCCTGCAAC |
| 1301 | TCGCCGGGGC CGATGTTCTG TTAGTCGATT CCGATCCCCA GGGCAGTGCC |
| 1351 | CGCGATTGGG CGGCCGTGCG GGAAGATCAA CCGCTAACCG TTGTCGGCAT |
| 1401 | CGACCGCCCG ACGATTGACC GCGACGTGAA GGCCATCGGC CGGCGCGACT |
| 1451 | TCGTAGTGAT CGACGGAGCG CCCCAGGCGG CGGACTTGGC TGTGTCCGCG |
| 1501 | ATCAAGGCAG CCGACTTCGT GCTGATTCCG GTGCAGCCAA GCCCTTACGA |
| 1551 | CATATGGGCC ACCGCCGACC TGGTGGAGCT GGTTAAGCAG CGCATTGAGG |
| 1601 | TCACGGATGG AAGGCTACAA GCGGCCTTTG TCGTGTCGCG GGCGATCAAA |
| 1651 | GGCACGCGCA TCGGCGGTGA GGTTGCCGAG GCGCTGGCCG GGTACGAGCT |
| 1701 | GCCCATTCTT GAGTCCCGTA TCACGCAGCG CGTGAGCTAC CCAGGCACTG |
| 1751 | CCGCCGCCGG CACAACCGTT CTTGAATCAG AACCCGAGGG CGACGCTGCC |
| 1801 | CGCGAGGTCC AGGCGCTGGC CGCTGAAATT AAATCAAAAC TCATTTGAGT |
| 1851 | TAATGAGGTA AAGAGAAAAT GAGCAAAAGC ACAAACACGC TAAGTGCCGG |
| 1901 | CCGTCCGAGC GCACGCAGCA GCAAGGCTGC AACGTTGGCC AGCCTGGCAG |
| 1951 | ACACGCCAGC CATGAAGCGG GTCAACTTTC AGTTGCCGGC GGAGGATCAC |
| 2001 | ACCAAGCTGA AGATGTACGC GGTACGCCAA GGCAAGACCA TTACCGAGCT |
| 2051 | GCTATCTGAA TACATCGCGC AGCTACCAGA GTAAATGAGC AAATGAATAA |
| 2101 | ATGAGTAGAT GAATTTTAGC GGCTAAAGGA GGCGGCATGG AAAATCAAGA |
| 2151 | ACAACCAGGC ACCGACGCCG TGGAATGCCC CATGTGTGGA GGAACGGGCG |
| 2201 | GTTGGCCAGG CGTAAGCGGC TGGGTTGTCT GCCGGCCCTG CAATGGCACT |
| 2251 | GGAACCCCCA AGCCCGAGGA ATCGGCGTGA CGGTCGCAAA CCATCCGGCC |
| 2301 | CGGTACAAAT CGGCGCGGCG CTGGGTGATG ACCTGGTGGA GAAGTTGAAG |
| 2351 | GCCGCGCAGG CCGCCCAGCG GCAACGCATC GAGGCAGAAG CACGCCCCGG |
| 2401 | TGAATCGTGG CAAGCGGCCG CTGATCGAAT CCGCAAAGAA TCCCGGCAAC |
| 2451 | CGCCGGCAGC CGGTGCGCCG TCGATTAGGA AGCCGCCCAA GGGCGACGAG |
| 2501 | CAACCAGATT TTTTCGTTCC GATGCTCTAT GACGTGGGCA CCCGCGATAG |
| 2551 | TCGCAGCATC ATGGACGTGG CCGTTTTCCG TCTGTCGAAG CGTGACCGAC |
| 2601 | GAGCTGGCGA GGTGATCCGC TACGAGCTTC CAGACGGGCA CGTAGAGGTT |
| 2651 | TCCGCAGGGC CGGCCGGCAT GGCCAGTGTG TGGGATTACG ACCTGGTACT |
| 2701 | GATGGCGGTT TCCCATCTAA CCGAATCCAT GAACCGATAC CGGGAAGGGA |
| 2751 | AGGGAGACAA GCCCGGCCGC GTGTTCCGTC CACACGTTGC GGACGTACTC |
| 2801 | AAGTTCTGCC GGCGAGCCGA TGGCGGAAAG CAGAAAGACG ACCTGGTAGA |
| 2851 | AACCTGCATT CGGTTAAACA CCACGCACGT TGCCATGCAG CGTACGAAGA |
| 2901 | AGGCCAAGAA CGGCCGCCTG GTGACGGTAT CCGAGGGTGA AGCCTTGATT |
| 2951 | AGCCGCTACA AGATCGTAAA GAGCGAAACC GGGCGGCCGG AGTACATCGA |
| 3001 | GATCGAGCTA GCTGATTGGA TGTACCGCGA GATCACAGAA GGCAAGAACC |
| 3051 | CGGACGTGCT GACGGTTCAC CCCGATTACT TTTTGATCGA TCCCGGCATC |
| 3101 | GGCCGTTTTC TCTACCGCCT GGCACGCCGC GCCGCAGGCA AGGCAGAAGC |
| 3151 | CAGATGGTTG TTCAAGACGA TCTACGAACG CAGTGGCAGC GCCGGAGAGT |
| 3201 | TCAAGAAGTT CTGTTTCACC GTGCGCAAGC TGATCGGGTC AAATGACCTG |
| 3251 | CCGGAGTACG ATTTGAAGGA GGAGGCGGGG CAGGCTGGCC CGATCCTAGT |
| 3301 | CATGCGCTAC CGCAACCTGA TCGAGGGCGA AGCATCCGCC GGTTCCTAAT |
| 3351 | GTACGGAGCA GATGCTAGGG CAAATTGCCC TAGCAGGGGA AAAAGGTCGA |
| 3401 | AAAGGTCTCT TTCCTGTGGA TAGCACGTAC ATTGGGAACC CAAAGCCGTA |
| 3451 | CATTGGGAAC CGGAACCCGT ACATTGGGAA CCCAAAGCCG TACATTGGGA |
| 3501 | ACCGGTCACA CATGTAAGTG ACTGATATAA AAGAGAAAAA AGGCGATTTT |
| 3551 | TCCGCCTAAA ACTCTTTAAA ACTTATTAAA ACTCTTAAAA CCCGCCTGGC |
| 3601 | CTGTGCATAA CTGTCTGGCC AGCGCACAGC CGAAGAGCTG CAAAAAGCGC |
| 3651 | CTACCCTTCG GTCGCTGCGC TCCCTACGCC CCGCCGCTTC GCGTCGGCCT |
| 3701 | ATCGCGGCCG CTGGCCGCTC AAAAATGGCT GGCCTACGGC CAGGCAATCT |
| 3751 | ACCAGGGCGC GGACAAGCCG CGCCGTCGCC ACTCGACCGC CGGCGCCCAC |
| 3801 | ATCAAGGCAC CCTGCCTCGC GCGTTTCGGT GATGACGGTG AAAACCTCTG |
| 3851 | ACACATGCAG CTCCCGGAGA CGGTCACAGC TTGTCTGTAA GCGGATGCCG |
| 3901 | GGAGCAGACA AGCCCGTCAG GGCGCGTCAG CGGGTGTTGG CGGGTGTCGG |
| 3951 | GGCGCAGCCA TGACCCAGTC ACGTAGCGAT AGCGGAGTGT ATACTGGCTT |
| 4001 | AACTATGCGG CATCAGAGCA GATTGTACTG AGAGTGCACC ATATGCGGTG |
| 4051 | TGAAATACCG CACAGATGCG TAAGGAGAAA ATACCGCATC AGGCGCTCTT |
| 4101 | CCGCTTCCTC GCTCACTGAC TCGCTGCGCT CGGTCGTTCG GCTGCGGCGA |
| 4151 | GCGGTATCAG CTCACTCAAA GGCGGTAATA CGGTTATCCA CAGAATCAGG |
| 4201 | GGATAACGCA GGAAAGAACA TGTGAGCAAA AGGCCAGCAA AAGGCCAGGA |
| 4251 | ACCGTAAAAA GGCCGCGTTG CTGGCGTTTT TCCATAGGCT CCGCCCCCCT |
| 4301 | GACGAGCATC ACAAAAATCG ACGCTCAAGT CAGAGGTGGC GAAACCCGAC |
| 4351 | AGGACTATAA AGATACCAGG CGTTTCCCCC TGGAAGCTCC CTCGTGCGCT |
| 4401 | CTCCTGTTCC GACCCTGCCG CTTACCGGAT ACCTGTCCGC CTTTCTCCCT |
| 4451 | TCGGGAAGCG TGGCGCTTTC TCATAGCTCA CGCTGTAGGT ATCTCAGTTC |
| 4501 | GGTGTAGGTC GTTCGCTCCA AGCTGGGCTG TGTGCACGAA CCCCCCGTTC |
| 4551 | AGCCCGACCG CTGCGCCTTA TCCGGTAACT ATCGTCTTGA GTCCAACCCG |
| 4601 | GTAAGACACG ACTTATCGCC ACTGGCAGCA GCCACTGGTA ACAGGATTAG |
| 4651 | CAGAGCGAGG TATGTAGGCG GTGCTACAGA GTTCTTGAAG TGGTGGCCTA |
| 4701 | ACTACGGCTA CACTAGAAGG ACAGTATTTG GTATCTGCGC TCTGCTGAAG |
| 4751 | CCAGTTACCT TCGGAAAAAG AGTTGGTAGC TCTTGATCCG GCAAACAAAC |
| 4801 | CACCGCTGGT AGCGGTGGTT TTTTTGTTTG CAAGCAGCAG ATTACGCGCA |
| 4851 | GAAAAAAAGG ATCTCAAGAA GATCCTTTGA TCTTTTCTAC GGGGTCTGAC |
| 4901 | GCTCAGTGGA ACGAAAACTC ACGTTAAGGG ATTTTGGTCA TGCATTCTAG |
| 4951 | GTACTAAAAC AATTCATCCA GTAAAATATA ATATTTTATT TTCTCCCAAT |
| 5001 | CAGGCTTGAT CCCCAGTAAG TCAAAAAATA GCTCGACATA CTGTTCTTCC |
| 5051 | CCGATATCCT CCCTGATCGA CCGGACGCAG AAGGCAATGT CATACCACTT |
| 5101 | GTCCGCCCTG CCGCTTCTCC CAAGATCAAT AAAGCCACTT ACTTTGCCAT |
| 5151 | CTTTCACAAA GATGTTGCTG TCTCCCAGGT CGCCGTGGGA AAAGACAAGT |
| 5201 | TCCTCTTCGG GCTTTTCCGT CTTTAAAAAA TCATACAGCT CGCGCGGATC |
| 5251 | TTTAAATGGA GTGTCTTCTT CCCAGTTTTC GCAATCCACA TCGGCCAGAT |
| 5301 | CGTTATTCAG TAAGTAATCC AATTCGGCTA AGCGGCTGTC TAAGCTATTC |
| 5351 | GTATAGGGAC AATCCGATAT GTCGATGGAG TGAAAGAGCC TGATGCACTC |
| 5401 | CGCATACAGC TCGATAATCT TTTCAGGGCT TTGTTCATCT TCATACTCTT |
| 5451 | CCGAGCAAAG GACGCCATCG GCCTCACTCA TGAGCAGATT GCTCCAGCCA |
| 5501 | TCATGCCGTT CAAAGTGCAG GACCTTTGGA ACAGGCAGCT TTCCTTCCAG |
| 5551 | CCATAGCATC ATGTCCTTTT CCCGTTCCAC ATCATAGGTG GTCCCTTTAT |
| 5601 | ACCGGCTGTC CGTCATTTTT AAATATAGGT TTTCATTTTC TCCCACCAGC |
| 5651 | TTATATACCT TAGCAGGAGA CATTCCTTCC GTATCTTTTA CGCAGCGGTA |
| 5701 | TTTTTCGATC AGTTTTTTCA ATTCCGGTGA TATTCTCATT TTAGCCATTT |
| 5751 | ATTATTTCCT TCCTCTTTTC TACAGTATTT AAAGATACCC CAAGAAGCTA |
| 5801 | ATTATAACAA GACGAACTCC AATTCACTGT TCCTTGCATT CTAAAACCTT |
| 5851 | AAATACCAGA AAACAGCTTT TTCAAAGTTG TTTTCAAAGT TGGCGTATAA |
| 5901 | CATAGTATCG ACGGAGCCGA TTTTGAAACC GCGGTGATCA CAGGCAGCAA |
| 5951 | CGCTCTGTCA TCGTTACAAT CAACATGCTA CCCTCCGCGA GATCATCCGT |
| 6001 | GTTTCAAACC CGGCAGCTTA GTTGCCGTTC TTCCGAATAG CATCGGTAAC |
| 6051 | ATGAGCAAAG TCTGCCGCCT TACAACGGCT CTCCCGCTGA CGCCGTCCCG |
| 6101 | GACTGATGGG CTGCCTGTAT CGAGTGGTGA TTTTGTGCCG AGCTGCCGGT |
| 6151 | CGGGGAGCTG TTGGCTGGCT GGTGGCAGGA TATATTGTGG TGTAAACAAA |
| 6201 | TTGACGCTTA GACAACTTAA TAACACATTG CGGACGTTTT TAATGTACTG |
| 6251 | AATTAACGCC GAATTAATTC GGGGGATCTG GATTTTAGTA CTGGATTTTG |
| 6301 | GTTTTAGGAA TTAGAAATTT TATTGATAGA AGTATTTTAC AAATACAAAT |
| 6351 | ACATACTAAG GGTTTCTTAT ATGCTCAACA CATGAGCGAA ACCCTATAGG |
| 6401 | AACCCTAATT CCCTTATCTG GGAACTACTC ACACATTATT ATGGAGAAAC |
| 6451 | TCGAGTCAAA TCTCGGTGAC GGGCAGGACC GGACGGGGCG GTACCGGCAG |
| 6501 | GCTGAAGTCC AGCTGCCAGA AACCCACGTC ATGCCAGTTC CCGTGCTTGA |
| 6551 | AGCCGGCCGC CCGCAGCATG CCGCGGGGGG CATATCCGAG CGCCTCGTGC |
| 6601 | ATGCGCACGC TCGGGTCGTT GGGCAGCCCG ATGACAGCGA CCACGCTCTT |
| 6651 | GAAGCCCTGT GCCTCCAGGG ACTTCAGCAG GTGGGTGTAG AGCGTGGAGC |
| 6701 | CCAGTCCCGT CCGCTGGTGG CGGGGGGAGA CGTACACGGT CGACTCGGCC |
| 6751 | GTCCAGTCGT AGGCGTTGCG TGCCTTCCAG GGGCCCGCGT AGGCGATGCC |
| 6801 | GGCGACCTCG CCGTCCACCT CGGCGACGAG CCAGGGATAG CGCTCCCGCA |
| 6851 | GACGGACGAG GTCGTCCGTC CACTCCTGCG GTTCCTGCGG CTCGGTACGG |
| 6901 | AAGTTGACCG TGCTTGTCTC GATGTAGTGG TTGACGATGG TGCAGACCGC |
| 6951 | CGGCATGTCC GCCTCGGTGG CACGGCGGAT GTCGGCCGGG CGTCGTTCTG |
| 7001 | GGCTCATGGT AGACTCGAGA GAGATAGATT TGTAGAGAGA GACTGGTGAT |
| 7051 | TTCAGCGTGT CCTCTCCAAA TGAAATGAAC TTCCTTATAT AGAGGAAGGT |
| 7101 | CTTGCGAAGG ATAGTGGGAT TGTGCGTCAT CCCTTACGTC AGTGGAGATA |
| 7151 | TCACATCAAT CCACTTGCTT TGAAGACGTG GTTGGAACGT CTTCTTTTTC |
| 7201 | CACGATGCTC CTCGTGGGTG GGGGTCCATC TTTGGGACCA CTGTCGGCAG |
| 7251 | AGGCATCTTG AACGATAGCC TTTCCTTTAT CGCAATGATG GCATTTGTAG |
| 7301 | GTGCCACCTT CCTTTTCTAC TGTCCTTTTG ATGAAGTGAC AGATAGCTGG |
| 7351 | GCAATGGAAT CCGAGGAGGT TTCCCGATAT TACCCTTTGT TGAAAAGTCT |
| 7401 | CAATAGCCCT TTGGTCTTCT GAGACTGTAT CTTTGATATT CTTGGAGTAG |
| 7451 | ACGAGAGTGT CGTGCTCCAC CATGTTATCA CATCAATCCA CTTGCTTTGA |
| 7501 | AGACGTGGTT GGAACGTCTT CTTTTTCCAC GATGCTCCTC GTGGGTGGGG |
| 7551 | GTCCATCTTT GGGACCACTG TCGGCAGAGG CATCTTGAAC GATAGCCTTT |
| 7601 | CCTTTATCGC AATGATGGCA TTTGTAGGTG CCACCTTCCT TTTCTACTGT |
| 7651 | CCTTTTGATG AAGTGACAGA TAGCTGGGCA ATGGAATCCG AGGAGGTTTC |
| 7701 | CCGATATTAC CCTTTGTTGA AAAGTCTCAA TAGCCCTTTG GTCTTCTGAG |
| 7751 | ACTGTATCTT TGATATTCTT GGAGTAGACG AGAGTGTCGT GCTCCACCAT |
| 7801 | GTTGGCAAGC TGCTCTAGCC AATACGCAAA CCGCCTCTCC CCGCGCGTTG |
| 7851 | GCCGATTCAT TAATGCAGCT GGCACGACAG GTTTCCCGAC TGGAAAGCGG |
| 7901 | GCAGTGAGCG CAACGCAATT AATGTGAGTT AGCTCACTCA TTAGGCACCC |
| 7951 | CAGGCTTTAC ACTTTATGCT TCCGGCTCGT ATGTTGTGTG GAATTGTGAG |
| 8001 | CGGATAACAA TTTCACACAG GAAACAGCTA TGACCATGAT TACGAATTGG |
| 8051 | GGTTTAAACC ACGGAAGATC CAGGTCTCGA GACTAGGAGA CGGATGGGAG |
| 8101 | GCGCAACGCG CGATGGGGAG GGGGGCGGCG CTGACCTTTC TGGCGAGGTC |
| 8151 | GAGGTAGCGA TCGAGCAGCT GCAGCGCGGA CACGATGAGG AAGACGAAGA |
| 8201 | TAGCCGCCAT GGACATGTTC GCCAGCGGCG GCGGAGCGAG GCTGAGCCGG |
| 8251 | TCTCTCCGGC CTCCGGTCGG CGTTAAGTTG GGGATCGTAA CGTGACGTGT |
| 8301 | CTCGTCTCCA CGGATCGACA CAACCGGCCT ACTCGGGTGC ACGACGCCGC |
| 8351 | GATAAGGGCG AGATGTCCGT GCACGCAGCC CGTTTGGAGT CCTCGTTGCC |
| 8401 | CACGAACCGA CCCCTTACAG AACAAGGCCT AGCCCAAAAC TATTCTGAGT |
| 8451 | TGAGCTTTTG AGCCTAGCCC ACCTAAGCCG AGCGTCATGA ACTGATGAAC |
| 8501 | CCACTACCAC TAGTCAAGGC AAACCACAAC CACAAATGGA TCAATTGATC |
| 8551 | TAGAACAATC CGAAGGAGGG GAGGCCACGT CACACTCACA CCAACCGAAA |
| 8601 | TATCTGCCAG AATCAGATCA ACCGGCCAAT AGGACGCCAG CGAGCCCAAC |
| 8651 | ACCTGGCGAC GCCGCAAAAT TCACCGCGAG GGGCACCGGG CACGGCAAAA |
| 8701 | ACAAAAGCCC GGCGCGGTGA GAATATCTGG CGACTGGCGG AGACCTGGTG |
| 8751 | GCCAGCGCGC GGCCACATCA GCCACCCCAT CCGCCCACCT CACCTCCGGC |
| 8801 | GAGCCAATGG CAACTCGTCT TAAGATTCCA CGAGATAAGG ACCCGATCGC |
| 8851 | CGGCGACGCT ATTTAGCCAG GTGCGCCCCC CACGGTACAC TCCACCAGCG |
| 8901 | GCATCTATAG CAACCGGTCC AGCACTTTCA CGCTCAGCTT CAGCAAGATC |
| 8951 | TACCGTCTTC GGTACGCGCT CACTCCGCCC TCTGCCTTTG TTACTGCCAC |
| 9001 | GTTTCTCTGA ATGCTCTCTT GTGTGGTGAT TGCTGAGAGT GGTTTAGCTG |
| 9051 | GATCTAGAAT TACACTCTGA AATCGTGTTC TGCCTGTGCT GATTACTTGC |
| 9101 | CGTCCTTTGT AGCAGCAAAA TATAGGGACA TGGTAGTACG AAACGAAGAT |
| 9151 | AGAACCTACA CAGCAATACG AGAAATGTGT AATTTGGTGC TTAGCGGTAT |
| 9201 | TTATTTAAGC ACATGTTGGT GTTATAGGGC ACTTGGATTC AGAAGTTTGC |
| 9251 | TGTTAATTTA GGCACAGGCT TCATACTACA TGGGTCAATA GTATAGGGAT |
| 9301 | TCATATTATA GGCGATACTA TAATAATTTG TTCGTCTGCA GAGCTTATTA |
| 9351 | TTTGCCAAAA TTAGATATTC CTATTCTGTT TTTGTTTGTG TGCTGTTAAA |
| 9401 | TTGTTAACGC CTGAAGGAAT AAATATAAAT GACGAAATTT TGATGTTTAT |
| 9451 | CTCTGCTCCT TTATTGTGAC CATAAGTCAA GATCAGATGC ACTTGTTTTA |
| 9501 | AATATTGTTG TCTGAAGAAA TAAGTACTGA CAGTATTTTG ATGCATTGAT |
| 9551 | CTGCTTGTTT GTTGTAACAA AATTTAAAAA TAAAGAGTTT CCTTTTTGTT |
| 9601 | GCTCTCCTTA CCTCCTGATG GTATCTAGTA TCTACCAACT GATACTATAT |
| 9651 | TGCTTCTCTT TACATACGTA TCTTGCTCGA TGCCTTCTCC TAGTGTTGAC |
| 9701 | CAGTGTTACT CACATAGTCT TTGCTCATTT CATTGTAATG CAGATACCAA |
| 9751 | GCGGCCTAGG AAAAATGGCT TCTATGATAT CCTCTTCCGC TGTGACAACA |
| 9801 | GTCAGCCGTG CCTCTAGGGG GCAATCCGCC GCAGTGGCTC CATTCGGCGG |
| 9851 | CCTCAAATCC ATGACTGGAT TCCCAGTGAA GAAGGTCAAC ACTGACATTA |
| 9901 | CTTCCATTAC AAGCAATGGT GGAAGAGTAA AGTGCATGCA GGTGTGGCCT |
| 9951 | CCAATTGGAA AGAAGAAGTT TGAGACTCTT TCCTATTTGC CACCATTGAC |
| 10001 | GAGAGATTCT AGAGTGAGTA ACAAGAACAA CGATGAGCTG CAGTGGCAAT |
| 10051 | CCTGGTTCAG CAAGGCGCCC ACCACCGAGG CGAACCCGAT GGCCACCATG |
| 10101 | TTGCAGGATA TCGGCGTTGC GCTCAAACCG GAAGCGATGG AGCAGCTGAA |
| 10151 | AAACGATTAT CTGCGTGACT TCACCGCGTT GTGGCAGGAT TTTTTGGCTG |
| 10201 | GCAAGGCGCC AGCCGTCAGC GACCGCCGCT TCAGCTCGGC AGCCTGGCAG |
| 10251 | GGCAATCCGA TGTCGGCCTT CAATGCCGCA TCTTACCTGC TCAACGCCAA |
| 10301 | ATTCCTCAGT GCCATGGTGG AGGCGGTGGA CACCGCACCC CAGCAAAAGC |
| 10351 | AGAAAATACG CTTTGCCGTG CAGCAGGTGA TTGATGCCAT GTCGCCCGCG |
| 10401 | AACTTCCTCG CCACCAACCC GGAAGCGCAG CAAAAACTGA TTGAAACCAA |
| 10451 | GGGCGAGAGC CTGACGCGTG GCCTGGTCAA TATGCTGGGC GATATCAACA |
| 10501 | AGGGCCATAT CTCGCTGTCG GACGAATCGG CCTTTGAAGT GGGCCGCAAC |
| 10551 | CTGGCCATTA CCCCGGGCAC CGTGATTTAC GAAAATCCGC TGTTCCAGCT |
| 10601 | GATCCAGTAC ACGCCGACCA CGCCGACGGT CAGCCAGCGC CCGCTGTTGA |
| 10651 | TGGTGCCGCC GTGCATCAAC AAGTTCTACA TCCTCGACCT GCAACCGGAA |
| 10701 | AATTCGCTGG TGCGCTACGC GGTGGAGCAG GGCAACACCG TGTTCCTGAT |
| 10751 | CTCGTGGAGC AATCCGGACA AGTCGCTGGC CGGCACCACC TGGGACGACT |
| 10801 | ACGTGGAGCA GGGCGTGATC GAAGCGATCC GCATCGTCCA GGACGTCAGC |
| 10851 | GGCCAGGACA AGCTGAACAT GTTCGGCTTC TGCGTGGGCG GCACCATCGT |
| 10901 | TGCCACCGCA CTGGCGGTAC TGGCGGCGCG TGGCCAGCAC CCGGCGGCCA |
| 10951 | GCCTGACCCT GCTGACCACC TTCCTCGACT TCAGCGACAC CGGCGTGCTC |
| 11001 | GACGTCTTCG TCGATGAAAC CCAGGTCGCG CTGCGTGAAC AGCAATTGCG |
| 11051 | CGATGGCGGC CTGATGCCGG GCCGTGACCT GGCCTCGACC TTCTCGAGCC |
| 11101 | TGCGTCCGAA CGACCTGGTA TGGAACTATG TGCAGTCGAA CTACCTCAAA |
| 11151 | GGCAATGAGC CGGCGGCGTT TGACCTGCTG TTCTGGAATT CGGACAGCAC |
| 11201 | CAATTTGCCG GGCCCGATGT TCTGCTGGTA CCTGCGCAAC ACCTACCTGG |
| 11251 | AAAACAGCCT GAAAGTGCCG GGCAAGCTGA CGGTGGCCGG CGAAAAGATC |
| 11301 | GACCTCGGCC TGATCGACGC CCCGGCCTTC ATCTACGGTT CGCGCGAAGA |
| 11351 | CCACATCGTG CCGTGGATGT CGGCGTACGG TTCGCTCGAC ATCCTCAACC |
| 11401 | AGGGCAAGCC GGGCGCCAAC CGCTTCGTGC TGGGCGCGTC CGGCCATATC |
| 11451 | GCCGGCGTGA TCAACTCGGT GGCCAAGAAC AAGCGCAGCT ACTGGATCAA |
| 11501 | CGACGGTGGC GCCGCCGATG CCCAGGCCTG GTTCGATGGC GCGCAGGAAG |
| 11551 | TGCCGGGCAG CTGGTGGCCG CAATGGGCCG GGTTCCTGAC CCAGCATGGC |
| 11601 | GGCAAGAAGG TCAAGCCCAA GGCCAAGCCC GGCAACGCCC GCTACACCGC |
| 11651 | GATCGAGGCG GCGCCCGGCC GTTACGTCAA AGCCAAGGGC TGAATGCAGG |
| 11701 | GATCCATCGT TCAAACATTT GGCAATAAAG TTTCTTAAGA TTGAATCCTG |
| 11751 | TTGCCGGTCT TGCGATGATT ATCATATAAT TTCTGTTGAA TTACGTTAAG |
| 11801 | CATGTAATAA TTAACATGTA ATGCATGACG TTATTTATGA GATGGGTTTT |
| 11851 | TATGATTAGA GTCCCGCAAT TATACATTTA ATACGCGATA GAAAACAAAA |
| 11901 | TATAGCGCGC AAACTAGGAT AAATTATCGC GCGCGGTGTC ATCTATGTTA |
| 11951 | CTAGATCCGA TGATAAGCTG TCAAACATGA GTTTAAACCA CGGAAGATCC |
| 12001 | AGGTCTCGAG ACTAGGAGAC GGATGGGAGG CGCAACGCGC GATGGGGAGG |
| 12051 | GGGGCGGCGC TGACCTTTCT GGCGAGGTCG AGGTAGCGAT CGAGCAGCTG |
| 12101 | CAGCGCGGAC ACGATGAGGA AGACGAAGAT AGCCGCCATG GACATGTTCG |
| 12151 | CCAGCGGCGG CGGAGCGAGG CTGAGCCGGT CTCTCCGGCC TCCGGTCGGC |
| 12201 | GTTAAGTTGG GGATCGTAAC GTGACGTGTC TCGTCTCCAC GGATCGACAC |
| 12251 | AACCGGCCTA CTCGGGTGCA CGACGCCGCG ATAAGGGCGA GATGTCCGTG |
| 12301 | CACGCAGCCC GTTTGGAGTC CTCGTTGCCC ACGAACCGAC CCCTTACAGA |
| 12351 | ACAAGGCCTA GCCCAAAACT ATTCTGAGTT GAGCTTTTGA GCCTAGCCCA |
| 12401 | CCTAAGCCGA GCGTCATGAA CTGATGAACC CACTACCACT AGTCAAGGCA |
| 12451 | AACCACAACC ACAAATGGAT CAATTGATCT AGAACAATCC GAAGGAGGGG |
| 12501 | AGGCCACGTC ACACTCACAC CAACCGAAAT ATCTGCCAGA ATCAGATCAA |
| 12551 | CCGGCCAATA GGACGCCAGC GAGCCCAACA CCTGGCGACG CCGCAAAATT |
| 12601 | CACCGCGAGG GGCACCGGGC ACGGCAAAAA CAAAAGCCCG GCGCGGTGAG |
| 12651 | AATATCTGGC GACTGGCGGA GACCTGGTGG CCAGCGCGCG GCCACATCAG |
| 12701 | CCACCCCATC CGCCCACCTC ACCTCCGGCG AGCCAATGGC AACTCGTCTT |
| 12751 | AAGATTCCAC GAGATAAGGA CCCGATCGCC GGCGACGCTA TTTAGCCAGG |
| 12801 | TGCGCCCCCC ACGGTACACT CCACCAGCGG CATCTATAGC AACCGGTCCA |
| 12851 | GCACTTTCAC GCTCAGCTTC AGCAAGATCT ACCGTCTTCG GTACGCGCTC |
| 12901 | ACTCCGCCCT CTGCCTTTGT TACTGCCACG TTTCTCTGAA TGCTCTCTTG |
| 12951 | TGTGGTGATT GCTGAGAGTG GTTTAGCTGG ATCTAGAATT ACACTCTGAA |
| 13001 | ATCGTGTTCT GCCTGTGCTG ATTACTTGCC GTCCTTTGTA GCAGCAAAAT |
| 13051 | ATAGGGACAT GGTAGTACGA AACGAAGATA GAACCTACAC AGCAATACGA |
| 13101 | GAAATGTGTA ATTTGGTGCT TAGCGGTATT TATTTAAGCA CATGTTGGTG |
| 13151 | TTATAGGGCA CTTGGATTCA GAAGTTTGCT GTTAATTTAG GCACAGGCTT |
| 13201 | CATACTACAT GGGTCAATAG TATAGGGATT CATATTATAG GCGATACTAT |
| 13251 | AATAATTTGT TCGTCTGCAG AGCTTATTAT TTGCCAAAAT TAGATATTCC |
| 13301 | TATTCTGTTT TTGTTTGTGT GCTGTTAAAT TGTTAACGCC TGAAGGAATA |
| 13351 | AATATAAATG ACGAAATTTT GATGTTTATC TCTGCTCCTT TATTGTGACC |
| 13401 | ATAAGTCAAG ATCAGATGCA CTTGTTTTAA ATATTGTTGT CTGAAGAAAT |
| 13451 | AAGTACTGAC AGTATTTTGA TGCATTGATC TGCTTGTTTG TTGTAACAAA |
| 13501 | ATTTAAAAAT AAAGAGTTTC CTTTTTGTTG CTCTCCTTAC CTCCTGATGG |
| 13551 | TATCTAGTAT CTACCAACTG ATACTATATT GCTTCTCTTT ACATACGTAT |
| 13601 | CTTGCTCGAT GCCTTCTCCT AGTGTTGACC AGTGTTACTC ACATAGTCTT |
| 13651 | TGCTCATTTC ATTGTAATGC AGATACCAAG CGGTTCGAAA AAAATGGCTT |
| 13701 | CTATGATATC CTCTTCCGCT GTGACAACAG TCAGCCGTGC CTCTAGGGGG |
| 13751 | CAATCCGCCG CAGTGGCTCC ATTCGGCGGC CTCAAATCCA TGACTGGATT |
| 13801 | CCCAGTGAAG AAGGTCAACA CTGACATTAC TTCCATTACA AGCAATGGTG |
| 13851 | GAAGAGTAAA GTGCATGCAG GTGTGGCCTC CAATTGGAAA GAAGAAGTTT |
| 13901 | GAGACTCTTT CCTATTTGCC ACCATTGACG AGAGATTCTA GAGTGACTGA |
| 13951 | CGTTGTCATC GTATCCGCCG CCCGCACCGC GGTCGGCAAG TTTGGCGGCT |
| 14001 | CGCTGGCCAA GATCCCGGCA CCGGAACTGG GTGCCGTGGT CATCAAGGCC |
| 14051 | GCGCTGGAGC GCGCCGGCGT CAAGCCGGAG CAGGTGAGCG AAGTCATCAT |
| 14101 | GGGCCAGGTG CTGACCGCCG GTTCGGGCCA GAACCCCGCA CGCCAGGCCG |
| 14151 | CGATCAAGGC CGGCCTGCCG GCGATGGTGC CGGCCATGAC CATCAACAAG |
| 14201 | GTGTGCGGCT CGGGCCTGAA GGCCGTGATG CTGGCCGCCA ACGCGATCAT |
| 14251 | GGCGGGCGAC GCCGAGATCG TGGTGGCCGG CGGCCAGGAA AACATGAGCG |
| 14301 | CCGCCCCGCA CGTGCTGCCG GGCTCGCGCG ATGGTTTCCG CATGGGCGAT |
| 14351 | GCCAAGCTGG TCGACACCAT GATCGTCGAC GGCCTGTGGG ACGTGTACAA |
| 14401 | CCAGTACCAC ATGGGCATCA CCGCCGAGAA CGTGGCCAAG GAATACGGCA |
| 14451 | TCACACGCGA GGCGCAGGAT GAGTTCGCCG TCGGCTCGCA GAACAAGGCC |
| 14501 | GAAGCCGCGC AGAAGGCCGG CAAGTTTGAC GAAGAGATCG TCCCGGTGCT |
| 14551 | GATCCCGCAG CGCAAGGGCG ACCCGGTGGC CTTCAAGACC GACGAGTTCG |
| 14601 | TGCGCCAGGG CGCCACGCTG GACAGCATGT CCGGCCTCAA GCCCGCCTTC |
| 14651 | GACAAGGCCG GCACGGTGAC CGCGGCCAAC GCCTCGGGCC TGAACGACGG |
| 14701 | CGCCGCCGCG GTGGTGGTGA TGTCGGCGGC CAAGGCCAAG GAACTGGGCC |
| 14751 | TGACCCCGCT GGCCACGATC AAGAGCTATG CCAACGCCGG TGTCGATCCC |
| 14801 | AAGGTGATGG GCATGGGCCC GGTGCCGGCC TCCAAGCGCG CCCTGTCGCG |
| 14851 | CGCCGAGTGG ACCCCGCAAG ACCTGGACCT GATGGAGATC AACGAGGCCT |
| 14901 | TTGCCGCGCA GGCGCTGGCG GTGCACCAGC AGATGGGCTG GGACACCTCC |
| 14951 | AAGGTCAATG TGAACGGCGG CGCCATCGCC ATCGGCCACC CGATCGGCGC |
| 15001 | GTCGGGCTGC CGTATCCTGG TGACGCTGCT GCACGAGATG AAGCGCCGTG |
| 15051 | ACGCGAAGAA GGGCCTGGCC TCGCTGTGCA TCGGCGGCGG CATGGGCGTG |
| 15101 | GCGCTGGCAG TCGAGCGCAA ATAACTGCAG GAGCTCATCG TTCAAACATT |
| 15151 | TGGCAATAAA GTTTCTTAAG ATTGAATCCT GTTGCCGGTC TTGCGATGAT |
| 15201 | TATCATATAA TTTCTGTTGA ATTACGTTAA GCATGTAATA ATTAACATGT |
| 15251 | AATGCATGAC GTTATTTATG AGATGGGTTT TTATGATTAG AGTCCCGCAA |
| 15301 | TTATACATTT AATACGCGAT AGAAAACAAA ATATAGCGCG CAAACTAGGA |
| 15351 | TAAATTATCG CGCGCGGTGT CATCTATGTT ACTAGATCCG ATGATAAGCT |
| 15401 | GTCAAACATG AGTTTAAACC ACGGAAGATC CAGGTCTCGA GACTAGGAGA |
| 15451 | CGGATGGGAG GCGCAACGCG CGATGGGGAG GGGGGCGGCG CTGACCTTTC |
| 15501 | TGGCGAGGTC GAGGTAGCGA TCGAGCAGCT GCAGCGCGGA CACGATGAGG |
| 15551 | AAGACGAAGA TAGCCGCCAT GGACATGTTC GCCAGCGGCG GCGGAGCGAG |
| 15601 | GCTGAGCCGG TCTCTCCGGC CTCCGGTCGG CGTTAAGTTG GGGATCGTAA |
| 15651 | CGTGACGTGT CTCGTCTCCA CGGATCGACA CAACCGGCCT ACTCGGGTGC |
| 15701 | ACGACGCCGC GATAAGGGCG AGATGTCCGT GCACGCAGCC CGTTTGGAGT |
| 15751 | CCTCGTTGCC CACGAACCGA CCCCTTACAG AACAAGGCCT AGCCCAAAAC |
| 15801 | TATTCTGAGT TGAGCTTTTG AGCCTAGCCC ACCTAAGCCG AGCGTCATGA |
| 15851 | ACTGATGAAC CCACTACCAC TAGTCAAGGC AAACCACAAC CACAAATGGA |
| 15901 | TCAATTGATC TAGAACAATC CGAAGGAGGG GAGGCCACGT CACACTCACA |
| 15951 | CCAACCGAAA TATCTGCCAG AATCAGATCA ACCGGCCAAT AGGACGCCAG |
| 16001 | CGAGCCCAAC ACCTGGCGAC GCCGCAAAAT TCACCGCGAG GGGCACCGGG |
| 16051 | CACGGCAAAA ACAAAAGCCC GGCGCGGTGA GAATATCTGG CGACTGGCGG |
| 16101 | AGACCTGGTG GCCAGCGCGC GGCCACATCA GCCACCCCAT CCGCCCACCT |
| 16151 | CACCTCCGGC GAGCCAATGG CAACTCGTCT TAAGATTCCA CGAGATAAGG |
| 16201 | ACCCGATCGC CGGCGACGCT ATTTAGCCAG GTGCGCCCCC CACGGTACAC |
| 16251 | TCCACCAGCG GCATCTATAG CAACCGGTCC AGCACTTTCA CGCTCAGCTT |
| 16301 | CAGCAAGATC TACCGTCTTC GGTACGCGCT CACTCCGCCC TCTGCCTTTG |
| 16351 | TTACTGCCAC GTTTCTCTGA ATGCTCTCTT GTGTGGTGAT TGCTGAGAGT |
| 16401 | GGTTTAGCTG GATCTAGAAT TACACTCTGA AATCGTGTTC TGCCTGTGCT |
| 16451 | GATTACTTGC CGTCCTTTGT AGCAGCAAAA TATAGGGACA TGGTAGTACG |
| 16501 | AAACGAAGAT AGAACCTACA CAGCAATACG AGAAATGTGT AATTTGGTGC |
| 16551 | TTAGCGGTAT TTATTTAAGC ACATGTTGGT GTTATAGGGC ACTTGGATTC |
| 16601 | AGAAGTTTGC TGTTAATTTA GGCACAGGCT TCATACTACA TGGGTCAATA |
| 16651 | GTATAGGGAT TCATATTATA GGCGATACTA TAATAATTTG TTCGTCTGCA |
| 16701 | GAGCTTATTA TTTGCCAAAA TTAGATATTC CTATTCTGTT TTTGTTTGTG |
| 16751 | TGCTGTTAAA TTGTTAACGC CTGAAGGAAT AAATATAAAT GACGAAATTT |
| 16801 | TGATGTTTAT CTCTGCTCCT TTATTGTGAC CATAAGTCAA GATCAGATGC |
| 16851 | ACTTGTTTTA AATATTGTTG TCTGAAGAAA TAAGTACTGA CAGTATTTTG |
| 16901 | ATGCATTGAT CTGCTTGTTT GTTGTAACAA AATTTAAAAA TAAAGAGTTT |
| 16951 | CCTTTTTGTT GCTCTCCTTA CCTCCTGATG GTATCTAGTA TCTACCAACT |
| 17001 | GATACTATAT TGCTTCTCTT TACANNNNNN TCTTGCTCGA TGCCTTCTCC |
| 17051 | TAGTGTTGAC CAGTGTTACT CACATAGTCT TTGCTCATTT CATTGTAATG |
| 17101 | CAGATACCAA GCGGTTAATA AAATGGCTTC TATGATATCC TCTTCCGCTG |
| 17151 | TGACAACAGT CAGCCGTGCC TCTAGGGGGC AATCCGCCGC AGTGGCTCCA |
| 17201 | TTCGGCGGCC TCAAATCCAT GACTGGATTC CCAGTGAAGA AGGTCAACAC |
| 17251 | TGACATTACT TCCATTACAA GCAATGGTGG AAGAGTAAAG TGCATGCAGG |
| 17301 | TGTGGCCTCC AATTGGAAAG AAGAAGTTTG AGACTCTTTC CTATTTGCCA |
| 17351 | CCATTGACGA GAGATTCTAG AGTGACTCAG CGCATTGCGT ATGTGACCGG |
| 17401 | CGGCATGGGT GGTATCGGAA CCGCCATTTG CCAGCGGCTG GCCAAGGATG |
| 17451 | GCTTTCGTGT GGTGGCCGGT TGCGGCCCCA ACTCGCCGCG CCGCGAAAAG |
| 17501 | TGGCTGGAGC AGCAGAAGGC CCTGGGCTTC GATTTCATTG CCTCGGAAGG |
| 17551 | CAATGTGGCT GACTGGGACT CGACCAAGAC CGCATTCGAC AAGGTCAAGT |
| 17601 | CCGAGGTCGG CGAGGTTGAT GTGCTGATCA ACAACGCCGG TATCACCCGC |
| 17651 | GACGTGGTGT TCCGCAAGAT GACCCGCGCC GACTGGGATG CGGTGATCGA |
| 17701 | CACCAACCTG ACCTCGCTGT TCAACGTCAC CAAGCAGGTG ATCGACGGCA |
| 17751 | TGGCCGACCG TGGCTGGGGC CGCATCGTCA ACATCTCGTC GGTGAACGGG |
| 17801 | CAGAAGGGCC AGTTCGGCCA GACCAACTAC TCCACCGCCA AGGCCGGCCT |
| 17851 | GCATGGCTTC ACCATGGCAC TGGCGCAGGA AGTGGCGACC AAGGGCGTGA |
| 17901 | CCGTCAACAC GGTCTCTCCG GGCTATATCG CCACCGACAT GGTCAAGGCG |
| 17951 | ATCCGCCAGG ACGTGCTCGA CAAGATCGTC GCGACGATCC CGGTCAAGCG |
| 18001 | CCTGGGCCTG CCGGAAGAGA TCGCCTCGAT CTGCGCCTGG TTGTCGTCGG |
| 18051 | AGGAGTCCGG TTTCTCGACC GGCGCCGACT TCTCGCTCAA CGGCGGCCTG |
| 18101 | CATATGGGCT GACTGCAGGG CGCCATCGTT CAAACATTTG GCAATAAAGT |
| 18151 | TTCTTAAGAT TGAATCCTGT TGCCGGTCTT GCGATGATTA TCATATAATT |
| 18201 | TCTGTTGAAT TACGTTAAGC ATGTAATAAT TAACATGTAA TGCATGACGT |
| 18251 | TATTTATGAG ATGGGTTTTT ATGATTAGAG TCCCGCAATT ATACATTTAA |
| 18301 | TACGCGATAG AAAACAAAAT ATAGCGCGCA AACTAGGATA AATTATCGCG |
| 18351 | CGCGGTGTCA TCTATGTTAC TAGATCCGAT GATAAGCTGT CAAACATGAT |
| 18401 | GTACAGTTTA AACCACGGAA GATCCAGGTC TCGAGACTAG GAGACGGATG |
| 18451 | GGAGGCGCAA CGCGCGATGG GGAGGGGGGC GGCGCTGACC TTTCTGGCGA |
| 18501 | GGTCGAGGTA GCGATCGAGC AGCTGCAGCG CGGACACGAT GAGGAAGACG |
| 18551 | AAGATAGCCG CCATGGACAT GTTCGCCAGC GGCGGCGGAG CGAGGCTGAG |
| 18601 | CCGGTCTCTC CGGCCTCCGG TCGGCGTTAA GTTGGGGATC GTAACGTGAC |
| 18651 | GTGTCTCGTC TCCACGGATC GACACAACCG GCCTACTCGG GTGCACGACG |
| 18701 | CCGCGATAAG GGCGAGATGT CCGTGCACGC AGCCCGTTTG GAGTCCTCGT |
| 18751 | TGCCCACGAA CCGACCCCTT ACAGAACAAG GCCTAGCCCA AAACTATTCT |
| 18801 | GAGTTGAGCT TTTGAGCCTA GCCCACCTAA GCCGAGCGTC ATGAACTGAT |
| 18851 | GAACCCACTA CCACTAGTCA AGGCAAACCA CAACCACAAA TGGATCAATT |
| 18901 | GATCTAGAAC AATCCGAAGG AGGGGAGGCC ACGTCACACT CACACCAACC |
| 18951 | GAAATATCTG CCAGAATCAG ATCAACCGGC CAATAGGACG CCAGCGAGCC |
| 19001 | CAACACCTGG CGACGCCGCA AAATTCACCG CGAGGGGCAC CGGGCACGGC |
| 19051 | AAAAACAAAA GCCCGGCGCG GTGAGAATAT CTGGCGACTG GCGGAGACCT |
| 19101 | GGTGGCCAGC GCGCGGCCAC ATCAGCCACC CCATCCGCCC ACCTCACCTC |
| 19151 | CGGCGAGCCA ATGGCAACTC GTCTTAAGAT TCCACGAGAT AAGGACCCGA |
| 19201 | TCGCCGGCGA CGCTATTTAG CCAGGTGCGC CCCCCACGGT ACACTCCACC |
| 19251 | AGCGGCATCT ATAGCAACCG GTCCAGCACT TTCACGCTCA GCTTCAGCAA |
| 19301 | GATCTACCGT CTTCGGTACG CGCTCACTCC GCCCTCTGCC TTTGTTACTG |
| 19351 | CCACGTTTCT CTGAATGCTC TCTTGTGTGG TGATTGCTGA GAGTGGTTTA |
| 19401 | GCTGGATCTA GAATTACACT CTGAAATCGT GTTCTGCCTG TGCTGATTAC |
| 19451 | TTGCCGTCCT TTGTAGCAGC AAAATATAGG GACATGGTAG TACGAAACGA |
| 19501 | AGATAGAACC TACACAGCAA TACGAGAAAT GTGTAATTTG GTGCTTAGCG |
| 19551 | GTATTTATTT AAGCACATGT TGGTGTTATA GGGCACTTGG ATTCAGAAGT |
| 19601 | TTGCTGTTAA TTTAGGCACA GGCTTCATAC TACATGGGTC AATAGTATAG |
| 19651 | GGATTCATAT TATAGGCGAT ACTATAATAA TTTGTTCGTC TGCAGAGCTT |
| 19701 | ATTATTTGCC AAAATTAGAT ATTCCTATTC TGTTTTTGTT TGTGTGCTGT |
| 19751 | TAAATTGTTA ACGCCTGAAG GAATAAATAT AAATGACGAA ATTTTGATGT |
| 19801 | TTATCTCTGC TCCTTTATTG TGACCATAAG TCAAGATCAG ATGCACTTGT |
| 19851 | TTTAAATATT GTTGTCTGAA GAAATAAGTA CTGACAGTAT TTTGATGCAT |
| 19901 | TGATCTGCTT GTTTGTTGTA ACAAAATTTA AAAATAAAGA GTTTCCTTTT |
| 19951 | TGTTGCTCTC CTTACCTCCT GATGGTATCT AGTATCTACC AACTGATACT |
| 20001 | ATATTGCTTC TCTTTACANN NNNNTCTTGC TCGATGCCTT CTCCTAGTGT |
| 20051 | TGACCAGTGT TACTCACATA GTCTTTGCTC ATTTCATTGT AATGCAGATA |
| 20101 | CCAAGCGGTT AATTAAAATG GCTTCTATGA TATCCTCTTC CGCTGTGACA |
| 20151 | ACAGTCAGCC GTGCCTCTAG GGGGCAATCC GCCGCAGTGG CTCCATTCGG |
| 20201 | CGGCCTCAAA TCCATGACTG GATTCCCAGT GAAGAAGGTC AACACTGACA |
| 20251 | TTACTTCCAT TACAAGCAAT GGTGGAAGAG TAAAGTGCAT GCAGGTGTGG |
| 20301 | CCTCCAATTG GAAAGAAGAA GTTTGAGACT CTTTCCTATT TGCCACCATT |
| 20351 | GACGAGAGAT TCTAGAGTGG AGAAGACGAT CGGTCTCGAG ATTATTGAAG |
| 20401 | TTGTCGAGCA GGCAGCGATC GCCTCGGCCC GCCTGATGGG CAAAGGCGAA |
| 20451 | AAGAATGAAG CCGATCGCGT CGCAGTAGAA GCGATGCGGG TGCGGATGAA |
| 20501 | CCAAGTGGAA ATGCTGGGCC GCATCGTCAT CGGTGAAGGC GAGCGCGACG |
| 20551 | AAGCACCGAT GCTCTATATC GGTGAAGAAG TGGGCATCTA CCGCGATGCA |
| 20601 | GACAAGCGGG CTGGCGTACC GGCTGGCAAG CTGGTGGAAA TCGACATCGC |
| 20651 | CGTTGACCCC TGCGAAGGCA CCAACCTCTG CGCCTACGGT CAGCCCGGCT |
| 20701 | CGATGGCAGT TTTGGCCATC TCCGAGAAAG GCGGCCTGTT TGCAGCTCCC |
| 20751 | GACTTCTACA TGAAGAAACT GGCTGCACCC CCAGCTGCCA AAGGCAAAGT |
| 20801 | AGACATCAAT AAGTCCGCGA CCGAAAACCT GAAAATTCTC TCGGAATGTC |
| 20851 | TCGATCGCGC CATCGATGAA TTGGTGGTCG TGGTCATGGA TCGTCCCCGC |
| 20901 | CACAAAGAGC TAATCCAAGA GATCCGCCAA GCGGGTGCCC GCGTCCGTCT |
| 20951 | GATCAGCGAT GGTGACGTTT CGGCCGCGAT CTCCTGCGGT TTTGCTGGCA |
| 21001 | CCAACACCCA CGCCCTGATG GGCATCGGTG CAGCTCCCGA GGGTGTGATT |
| 21051 | TCGGCAGCAG CAATGCGTTG CCTCGGCGGT CACTTCCAAG GCCAGCTGAT |
| 21101 | CTACGACCCA GAAGTGGTCA AAACCGGCCT GATCGGTGAA AGCCGTGAGA |
| 21151 | GCAACATCGC TCGCCTGCAA GAAATGGGCA TCACCGATCC CGATCGCGTC |
| 21201 | TACGACGCCA ACGAACTGGC TTCGGGTCAA GAAGTGCTGT TTGCGGCTTG |
| 21251 | CGGTATCACC CCGGGCTTGC TGATGGAAGG CGTGCGCTTC TTCAAAGGCG |
| 21301 | GCGCTCGCAC CCAGAGCTTG GTGATCTCCA GCCAGTCACG GACGGCTCGC |
| 21351 | TTCGTTGACA CCGTTCACAT GTTCGACGAT GTCAAAACGG TTAGCCTCCG |
| 21401 | TTAAGGCGCG CCATCGTTCA AACATTTGGC AATAAAGTTT CTTAAGATTG |
| 21451 | AATCCTGTTG CCGGTCTTGC GATGATTATC ATATAATTTC TGTTGAATTA |
| 21501 | CGTTAAGCAT GTAATAATTA ACATGTAATG CATGACGTTA TTTATGAGAT |
| 21551 | GGGTTTTTAT GATTAGAGTC CCGCAATTAT ACATTTAATA CGCGATAGAA |
| 21601 | AACAAAATAT AGCGCGCAAA CTAGGATAAA TTATCGCGCG CGGTGTCATC |
| 21651 | TATGTTACTA GATCCGATGA TAAGCTGTCA AACATGACCT CAGGATGAAG |
| 21701 | CTTGGCACTG GCCGTCGTTT TACAACGTCG TGACTGGGAA AACCCTGGCG |
| 21751 | TTACCCAACT TAATCGCCTT GCAGCACATC CCCCTTTCGC CAGCTGGCGT |
| 21801 | AATAGCGAAG AGGCCCGCAC CGATCGCCCT TCCCAACAGT TGCGCAGCCT |
| 21851 | GAATGGCGAA TGCTAGAGCA GCTTGAGCTT GGATCAGATT GTCGTTTCCC |
| 21901 | GCCTTCAGTT TAAACTATCA GTGTTTGACA GGATATATTG GCGGGTAAAC |
| 21951 | CTAAGAGAAA AGAGCGTTTA TTAGAATAAC GGATATTTAA AAGGGCGTGA |
| 22001 | AAAGGTTTAT CCGTTCGTCC ATTTGTATGT G |

We claim:

1. A transgenic plant or transgenic plant cell genetically engineered to produce polyhydroxyalkanoate, wherein the transgenic plant or plant cell produces increased lignocellulosic biomass relative to a corresponding non-genetically-engineered plant or plant cell.

2. The transgenic plant or transgenic plant cell of claim 1 wherein the transgenic plant or transgenic plant cell comprises the NAD-malic enzyme photosynthetic pathway.

3. The transgenic plant or transgenic plant cell of claim 1, wherein the transgenic plant or transgenic plant cell further comprises one or more transgenes that increase carbon flow for the production of polyhydroxyalkanoates.

4. The transgenic plant or transgenic plant cell of claim 3 wherein the one or more transgenes increase carbon flow through the Calvin cycle in photosynthesis.

5. The transgenic plant or transgenic plant cell of claim 4 wherein the one or more transgenes that increase carbon flow through the Calvin cycle are selected from the group consisting of sedoheptulose 1,7-bisphosphatase (SBPase, EC 3.1.3.37), fructose 1,6-bisphosphatase (FBPase, EC 3.1.3.11), a bi-functional enzyme with both SBPase and FBPase activities, transketolase (EC 2.2.1.1), and aldolase (EC 4.1.2.13).

6. The transgenic plant or transgenic plant cell of claim 5 wherein the bifunctional enzyme is selected from the group consisting of Ralstonia eutropha H16 (Accession number AAA69974), Synechococcus elongatus PCC 7942 (Accession numbers D83512 (SEQ ID NO: 2) and CP000100 (SEQ ID NO: 1)), Synechococcus sp. WH 7805 (Accession number ZP_{—}01124026), Butyrivibrio crossotus DSM 2876 (Accession number EFF67670), Rothia mucilaginosa DY-18 (Accession number YP 003363264), Thiobacillus denitrificans ATCC 25259 (Accession number AAZ98530), Methylacidiphilum infernorum V4 (Accession number ACD83413), Nitrosomonas europaea ATCC 19718 (Accession number CAD84432), Vibrio vulnificus CMCP6 (Accession number AA009802), and Methanohalophilus mahii DSM 5219 (Accession number YP_{—}003542799).

7. The transgenic plant or transgenic plant cell of claim 6 wherein the plant or plant cell transformed to produce the transgenic plant or transgenic plant cell is selected from the group consisting of switchgrass, Miscanthus, Sorghum, sugarcane, energy cane, giant reed, millets, Napier grass, other forage grasses and turf grasses.

8. The transgenic plant or transgenic plant cell of claim 7 wherein the plant is switchgrass (Panicum virgatum L.).

9. The transgenic plant or transgenic plant cell of claim 8 wherein the cultivar of switchgrass is Alamo.

10. The transgenic plant or transgenic plant cell of claim 8 wherein the cultivar of switchgrass is selected from the group consisting of Blackwell, Kanlow, Nebraska 28, Pathfinder, Cave-in-Rock, Shelter and Trailblazer.

11. The transgenic plant or transgenic plant cell of claim 1 wherein the plant transformed to produce the transgenic plant is a C₄ plant.

12. The transgenic plant of claim 1 wherein the transgenic plant produces at least about 4% dry weight (dwt) polyhydroxyalkanoate.

13. The transgenic plant of claim 12 wherein the transgenic plant produces at least about 5% dry weight (dwt) polyhydroxyalkanoate.

14. The transgenic plant of claim 12 wherein the transgenic plant produces at least about 6% dry weight (dwt) polyhydroxyalkanoate.

15. The transgenic plant of claim 12 wherein the transgenic plant produces at least about 7% dry weight (dwt) polyhydroxyalkanoate.

16. The transgenic plant of claim 12 wherein the transgenic plant produces at least about 8% dry weight (dwt) polyhydroxyalkanoate.

17. A transgenic plant produced from the transgenic plants or transgenic plant cells of claim 1.

18. A seed obtained from the transgenic plant of claim 1.

19. A feedstock composition for production of biofuel, pyrolysis liquids, syngas, steam power or cogeneration power, wherein the feedstock comprises at least about 3 to about 7.7% PHB and lignocellulosic biomass.

20. A feedstock composition for production of biofuel, pyrolysis liquids, syngas, steam power or cogeneration power, wherein the feedstock comprises at least about 3 to about 7.7% PHB and lignocellulosic biomass with modified structural carbohydrates.

21. The feedstock composition of claim 19, wherein feedstock is obtained from the transgenic plant of claim 1.

22. A method for increasing carbon flow through the Calvin cycle in photosynthesis, the method comprising:
introducing into the embryogenic callus cultures initiated from a transgenic plant transgenes that increase carbon flow through the Calvin cycle, thereby producing re-transformed callus cultures; and
regenerating plants from the re-transformed callus cultures, thereby producing plants with increased carbon flow through the Calvin cycle in photosynthesis;
wherein the transgenes that increase carbon flow through the Calvin cycle are selected from the group consisting of sedoheptulose 1,7-bisphosphatase (SBPase, EC 3.1.3.37), fructose 1,6-bisphosphatase (FBPase, EC 3.1.3.11), a bi-functional enzyme with both SBPase and FBPase activities, transketolase (EC 2.2.1.1), and aldolase (EC 4.1.2.13).

23. The method of claim 22, wherein the bifunctional enzyme is selected from the group consisting of Ralstonia eutropha H16 (Accession number AAA69974), Synechococcus elongatus PCC 7942 (Accession numbers D83512 (SEQ ID NO: 2) and CP000100 (SEQ ID NO: 1)), Synechococcus sp. WH 7805 (Accession number ZP_{—}01124026), Butyrivibrio crossotus DSM 2876 (Accession number EFF67670), Rothia mucilaginosa DY-18 (Accession number YP_{—}003363264), Thiobacillus denitrificans ATCC 25259 (Accession number AAZ98530), Methylacidiphilum infernorum V4 (Accession number ACD83413), Nitrosomonas europaea ATCC 19718 (Accession number CAD84432), Vibrio vulnificus CMCP6 (Accession number AA009802), and Methanohalophilus mahii DSM 5219 (Accession number YP_{—}003542799).

24. The method of claim 23, wherein the embryogenic callus culture is derived from a plant selected from the group consisting of switchgrass, Miscanthus, Sorghum, sugarcane, energy cane, giant reed, millets, Napier grass, other forage grasses and turf grasses.

25. The method of claim 24, wherein the plant is switchgrass (Panicum virgatum L.).

26. The method of claim 25, wherein the plant is the Alamo cultivar of switchgrass.

27. The method of claim 25, wherein the plant is a cultivar of switchgrass selected from the group consisting of Blackwell, Kanlow, Nebraska 28, Pathfinder, Cave-in-Rock, Shelter and Trailblazer.

28. The method of claim 22, wherein the embryogenic callus culture is derived from a transgenic C₄ plant.

29. The method of claim 28, wherein the plants with increased carbon flow through the Calvin cycle in photosynthesis produce at least about 4% dry weight (dwt) polyhydroxyalkanoate.

30. The method of claim 28, wherein the plants with increased carbon flow through the Calvin cycle in photosynthesis produce at least about 5% dry weight (dwt) polyhydroxyalkanoate.

31. The method of claim 28, wherein the plants with increased carbon flow through the Calvin cycle in photosynthesis produce at least about 6% dry weight (dwt) polyhydroxyalkanoate.

32. The method of claim 28, wherein the plants with increased carbon flow through the Calvin cycle in photosynthesis produce at least about 7% dry weight (dwt) polyhydroxyalkanoate.

33. The method of claim 28, wherein the plants with increased carbon flow through the Calvin cycle in photosynthesis produce at least about 8% dry weight (dwt) polyhydroxyalkanoate.


SEQUENCE LISTING (6 sequences)

SEQ ID NO: 1 - protein, 265 aa, Synechococcus elongatus
RAGVPAGKLVEIDIAVDPCEGTNLCAYGQPGSMAVLAISEKGGLFAAPDFYMKKLAAPPAAKGKVDINKSATENLKILSECLDRAIDELVVVVMDRPRHKELIQEIRQAGARVRLISDGDVSAAISCGFAGTNTHALMGIGAAPEGVISAAAMRCLGGHFQGQLIYDPEVVKTGLIGESRESNIARLQEMGITDPDRVYDANELASGQEVLFAACGITPGLLMEGVRFFKGGARTQSLVISSQSRTARFVDTVHMFDDVKTVSLR

SEQ ID NO: 2 - protein, 276 aa, Synechococcus elongatus
RAGVPAGKLVEIDIAVDPCEGTNLCAYGQPGSMAVLAISEKGGLFAAPDFYMKKLAAPPAAKGKETSIKSATENLKILSECLDRAIDELVVVVMDRPRHKELIQEIRQAGARVRLISDGDVSAAISCGFAGTNTHALMGIGAAPEGVISAAAMRCLGGHFQGQLIYDPEVVKTGLIGESRESNIARLQEMGITDPDRVYDANELASGQEVLFAACGITPGLLMEGVRFFKGGARTQSLVISSQSRTARFVDTVHMFDDVKTVSLPLIPDPKWRPER

SEQ ID NO: 3 - DNA, 13180 bp, Artificial Sequence
  Synthetic Vector
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagttatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttacctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatggtagaggagctcgccgcttggtatctgcattacaatgaaatgagcaaagactatgtgagtaacactggtcaacactagggagaaggcatcgagcaagatacgtatgtaaagagaagcaatatagtgtcagttggtagatactagataccatcaggaggtaaggagagcaacaaaaaggaaactctttatttttaaattttgttacaacaaacaagcagatcaatgcatcaaaatactgtcagtacttatttcttcagacaacaatatttaaaacaagtgcatctgatcttgacttatggtcacaataaaggagcagagataaacatcaaaatttcgtcatttatatttattccttcaggcgttaacaatttaacagcacacaaacaaaaacagaataggaatatctaattttggcaaataataagctctgcagacgaacaaattattatagtatcgcctataatatgaatccctatactattgacccatgtagtatgaagcctgtgcctaaattaacagcaaacttctgaatccaagtgccctataacaccaacatgtgcttaaataaataccgctaagcaccaaattacacatttctcgtattgctgtgtaggttctatcttcgtttcgtactaccatgtccctatattttgctgctacaaaggacggcaagtaatcagcacaggcagaacacgatttcagagtgtaattctagatccagctaaaccactctcagcaatcaccacacaagagagcattcagagaaacgtggcagtaacaaaggcagagggcggagtgagcgcgtaccgaagacggtagatctctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtaccccacggaagatccaggtctcgagactaggagacggatgggaggcgcaacgcgcgatggggaggggggcggcgctgacctttctggcgaggtcgaggtagcgatcgagcagctgcagcgcggacacgatgaggaagacgaagatagccgccatggacatgttcgccagcggcggcggagcgaggctgagccggtctctccggcctccggtcggcgttaagttggggatcgtaacgtgacgtgtctcgtctccacggatcgacacaaccggcctactcgggtgcacgacgccgcgataagggcgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagaatcagatcaaccggccaataggacgccagcgagcccaacacctggcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccagcactttcacgctcagcttcagcaagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgatactatattgcttctctttacannnnnntcttgctcgatgccttctcctagtgttgaccagtgttactcacatagtctttgctcatttcattgtaatgcagataccaagcggttaattaaaaatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgcaggtgtggcctccaattggaaagaagaagtttgagactctttcctatttgccaccattgacgagagattctagagtggagaagacgatcggtctcgagattattgaagttgtcgagcaggcagcgatcgcctcggcccgcctgatgggcaaaggcgaaaagaatgaagccgatcgcgtcgcagtagaagcgatgcgggtgcggatgaaccaagtggaaatgctgggccgcatcgtcatcggtgaaggcgagcgcgacgaagcaccgatgctctatatcggtgaagaagtgggcatctaccgcgatgcagacaagcgggctggcgtaccggctggcaagctggtggaaatcgacatcgccgttgacccctgcgaaggcaccaacctctgcgcctacggtcagcccggctcgatggcagttttggccatctccgagaaaggcggcctgtttgcagctcccgacttctacatgaagaaactggctgcacccccagctgccaaaggcaaagtagacatcaataagtccgcgaccgaaaacctgaaaattctctcggaatgtctcgatcgcgccatcgatgaattggtggtcgtggtcatggatcgtccccgccacaaagagctaatccaagagatccgccaagcgggtgcccgcgtccgtctgatcagcgatggtgacgtttcggccgcgatctcctgcggttttgctggcaccaacacccacgccctgatgggcatcggtgcagctcccgagggtgtgatttcggcagcagcaatgcgttgcctcggcggtcacttccaaggccagctgatctacgacccagaagtggtcaaaaccggcctgatcggtgaaagccgtgagagcaacatcgctcgcctgcaagaaatgggcatcaccgatcccgatcgcgtctacgacgccaacgaactggcttcgggtcaagaagtgctgtttgcggcttgcggtatcaccccgggcttgctgatggaaggcgtgcgcttcttcaaaggcggcgctcgcacccagagcttggtgatctccagccagtcacggacggctcgcttcgttgacaccgttcacatgttcgacgatgtcaaaacggttagcctccgttaactgcagggcgcgccatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 4 - protein, 14 aa, Artificial Sequence
  Synthetic peptide
LMGKGEKNEADRVA

SEQ ID NO: 5 - DNA, 30 bp, Artificial Sequence
  Synthetic Primer
ccgaattcgtggagaagacgatcggtctcg

SEQ ID NO: 6 - DNA, 30 bp, Artificial Sequence
  Synthetic Primer
cctctagactaccgctccggccgccatttg